SEQUENCE LISTING (12 sequences)

SEQ ID NO: 1 - DNA, 2024 bp, Triticum aestivum
ctggcttgctcattttgcggtagtgtttaaacatcggctagccttacgggtataaaaaggtgggcaacttcaccctatcccatagcactagaccaaagaacacctatactccatactacccttcgttcacctggtgagcttcttcttcctttgatctatatcacttactatttctcccttgtccagcttcttcttcttcctcgtgcatgcgactttttctagataatatcccgcactatcgctcgccgcaagatgtgctagctagcgatcttcactttaatacctgttgtagatctaaccacgggctattccaaaaaatatttgtcttgtttgcgtgttcctgtgtacatgcacgtatctagatcttgattttgaagaattcataattaattcatgacctaccttgtttggtttgtgtaattttgatgttgtcgtatcaattttagcaaaccactcgtagctagaacaatagagggggcgatcgtatgtttctgttttgaaaaggggatatttccaggctctgcatcggttcatgcacacagccgttaccacattcaataggcactgatccatggatgcatgccagatttactagttttgtatacaaagttttacttttttgctttgatttatgaaaagttggatcagattttgcagttctcttttatccatgttggattcactactttgtacccaagattttatttattttgtcttggtttcttacctgcctggttagtaactaggagatcctgggattagactttcaaggaatcctaatactagtgagtatagggaaaggaagcttatttttaagctgcccaaaagaatgggcgcttagagttgtagttgattaattgaatctgttctgtggatttgagaatttcagacctgattctacatgacattttgagttaaccaatgattctacatgtctcactccttgggattaacaatttaactttatttaattcgatatgtgtgtacacatgtgttgcagatggaggaagtgtttccgtcaaactccaagagcaaggccggtcagatggcgggggaggcgacagcggcggcggagaagaagcctcggccgaagccagagcagaaggtggaatgccctcggtgcaagtctggcaacaccaagttctgctactacaacaactatagtatgtctcagccccgctacttctgcaaggcctgccgccgctactggacccatggtggctccctccgcaacgtccccatcggtggcggctgccgcaagcccaagcgcccggggacctccgacgcccacaagctcggcgtggcctcctcgtcagaacccacggttgtcatgccgccctcgacctgcacagggatgaactttgccaacgtcctcccaacatttatgtctgctggttttgagattccaagcagcctttccctgactgcctttgggtcatcgtcatcgtccaacacggcggcagtgatgtcccctggtgggacgacgtcatttctagacgtgttgagagggggcgcaggagggcttcttgatggcagcctcagtcagaacaatggctactactatggtgggcctgccactggatcaggcattgggatgctgatgacgccgccagtggcgtcatttggcattccaggtccgatgcagcaacatggtgatctcgtggttggtggaaatggaataggtgctgcaactgcttcaatatttcaggggggcactggcgaggaaggagatgatggtacggggggcgtgatggggctccaatggcagccacaggttggcaatggtggaggtgctggtgttgtatcaggaggcgtgcatcaccttgggactgggaacaatgtgacgatgggcaacaacaatatacacaacaacaacaataacaacagtgggggtgatgacaacaatggtgcgtcatcgagggattgctactggatcaacaatggaggatcgaacccatggcagagcctcctcaacaacagctccctgatgtaagtgcaataagaaaatgggaaatggaggtcat

SEQ ID NO: 2 - DNA, 990 bp, Triticum aestivum
atggaggaagtgtttccgtcaaactccaagagcaaggccggtcagatggcgggggaggcgacagcggcggcggagaagaagcctcggccgaagccagagcagaaggtggaatgccctcggtgcaagtctggcaacaccaagttctgctactacaacaactatagtatgtctcagccccgctacttctgcaaggcctgccgccgctactggacccatggtggctccctccgcaacgtccccatcggtggcggctgccgcaagcccaagcgcccggggacctccgacgcccacaagctcggcgtggcctcctcgtcagaacccacggttgtcatgccgccctcgacctgcacagggatgaactttgccaacgtcctcccaacatttatgtctgctggttttgagattccaagcagcctttccctgactgcctttgggtcatcgtcatcgtccaacacggcggcagtgatgtcccctggtgggacgacgtcatttctagacgtgttgagagggggcgcaggagggcttcttgatggcagcctcagtcagaacaatggctactactatggtgggcctgccactggatcaggcattgggatgctgatgacgccgccagtggcgtcatttggcattccaggtccgatgcagcaacatggtgatctcgtggttggtggaaatggaataggtgctgcaactgcttcaatatttcaggggggcactggcgaggaaggagatgatggtacggggggcgtgatggggctccaatggcagccacaggttggcaatggtggaggtgctggtgttgtatcaggaggcgtgcatcaccttgggactgggaacaatgtgacgatgggcaacaacaatatacacaacaacaacaataacaacagtgggggtgatgacaacaatggtgcgtcatcgagggattgctactggatcaacaatggaggatcgaacccatggcagagcctcctcaacaacagctccctgatg

SEQ ID NO: 3 - protein, 330 aa, Triticum aestivum
MEEVFPSNSKSKAGQMAGEATAAAEKKPRPKPEQKVECPRCKSGNTKFCYYNNYSMSQPRYFCKACRRYWTHGGSLRNVPIGGGCRKPKRPGTSDAHKLGVASSSEPTVVMPPSTCTGMNFANVLPTFMSAGFEIPSSLSLTAFGSSSSSNTAAVMSPGGTTSFLDVLRGGAGGLLDGSLSQNNGYYYGGPATGSGIGMLMTPPVASFGIPGPMQQHGDLVVGGNGIGAATASIFQGGTGEEGDDGTGGVMGLQWQPQVGNGGGAGVVSGGVHHLGTGNNVTMGNNNIHNNNNNNSGGDDNNGASSRDCYWINNGGSNPWQSLLNNSSLM

SEQ ID NO: 4 - DNA, 2028 bp, Triticum aestivum
ctggcttgctcattttgcggtagtgtttaaacattggctggaattacgggtataaaaaggagggcaacttcaccctatcccatagcactagaccaaacaactcctatactccatactacccttcattcacctggtgagcttcttctttctttgatttctatcacttactctttctccctcgtccagcttcttcttcttcctcgtgcatgtgacttttgctagataatctcccacattatcgctcaatgcaagccgtgctagctagctagcgatctagctagcgatcttcactttaatacccgttgtagatctaaccatgggctattccaaaacatatttctcttgtttgcgtgttcgtgtgtacatgcatgcatctagatcttgattttgaggaattcataagtaattcctgacctaccttgtttggtttgtttaattttgatgttgttgtctcaattttagcaaattgctcgtagctagaacaatagagggggcggccgtatgtttccgttttgaaaaggggatatttccaggctctgcatcggttgatgcacacagccgttaccacattcaataggcactgatccatggatgcatgctatatttacaagttttctatagaaattttttttatttatgaaaaattggatcggtatagttcttctttatccatgtcggattcactactttgtacccaagattttatttattttgtctcggtttcttacatgtctagttaggtaactaggagagcctgggattaggctttcaaggaatcctaatactagagactatggggagagacagcttattctttaagctgcgcaaaagaatgggcgcttagagttgtagttgataaattgaatctgttgtatggatttgagaatttgagacctgattatgcacttatcatgaaattttgagttaaccaatgattctacatgtctcactccttaggattaacaatttaacttaatttaattcgatatgtgtgtacacatgtgttgaagatggaggaagtgtttccgtcaaactccaagagcaaggctggtcagatggcgggggaggcgacagcggcggcggagaagaagcctcggccgaagccagagcagaaggtggaatgccctcggtgcaagtctggcaacaccaagttctgctactacaacaactatagtatgtctcagccccgctacttctgcaaggcctgccgccgctactggacccatggtgggtccctccgtaacgtccccatcggtggtggctgccgcaagcccaagcgctcggggacctccgacgcccacaagctcggcgtggcctcctcgtcggaacacacggctgtcatgcccccctcgacctgcacagggataaactttgccaatgtcctcccgacgtttatgtctgctggttttgagattccaagaagcctttccctgaccacctttgggtcatcgtcgtcgtccaacacgacggctgtcatgtcccctggtgggacgacgtcatttctagacgtgctgagagggggaacaggagggcttcttgatggcaacctcggtcagaacaatggctactactatggtgggtctagatcaggcattgggatgctgatgacgccgccagcggcgtcatttggcattccaggtccaatgcagcagcatggcgatctcatggttggtggaaatggaataggtgccgcaactgcttcaatatttcaggggggcactggtgaggaaggagatgacggcaaaggggccatgatggggctccaatggcagccacatgttggtaatggtggaggtggtggtgttgtatcaggaggcgtgcatcaccttgggactgggaacaatgtgacgatgggcaacaacaacataaacaacaataacaataatggcagccacagtgatgacaacactggtgggtcatcgagggattgctactggatcaataatggaggatcgaacccatggcaaagcctcctcaatagcagctccctgatgtaagtgcaagaagaaaatgcgaaatggagatcat

SEQ ID NO: 5 - DNA, 984 bp, Triticum aestivum
atggaggaagtgtttccgtcaaactccaagagcaaggctggtcagatggcgggggaggcgacagcggcggcggagaagaagcctcggccgaagccagagcagaaggtggaatgccctcggtgcaagtctggcaacaccaagttctgctactacaacaactatagtatgtctcagccccgctacttctgcaaggcctgccgccgctactggacccatggtgggtccctccgtaacgtccccatcggtggtggctgccgcaagcccaagcgctcggggacctccgacgcccacaagctcggcgtggcctcctcgtcggaacacacggctgtcatgcccccctcgacctgcacagggataaactttgccaatgtcctcccgacgtttatgtctgctggttttgagattccaagaagcctttccctgaccacctttgggtcatcgtcgtcgtccaacacgacggctgtcatgtcccctggtgggacgacgtcatttctagacgtgctgagagggggaacaggagggcttcttgatggcaacctcggtcagaacaatggctactactatggtgggtctagatcaggcattgggatgctgatgacgccgccagcggcgtcatttggcattccaggtccaatgcagcagcatggcgatctcatggttggtggaaatggaataggtgccgcaactgcttcaatatttcaggggggcactggtgaggaaggagatgacggcaaaggggccatgatggggctccaatggcagccacatgttggtaatggtggaggtggtggtgttgtatcaggaggcgtgcatcaccttgggactgggaacaatgtgacgatgggcaacaacaacataaacaacaataacaataatggcagccacagtgatgacaacactggtgggtcatcgagggattgctactggatcaataatggaggatcgaacccatggcaaagcctcctcaatagcagctccctgatg

SEQ ID NO: 6 - protein, 328 aa, Triticum aestivum
MEEVFPSNSKSKAGQMAGEATAAAEKKPRPKPEQKVECPRCKSGNTKFCYYNNYSMSQPRYFCKACRRYWTHGGSLRNVPIGGGCRKPKRSGTSDAHKLGVASSSEHTAVMPPSTCTGINFANVLPTFMSAGFEIPRSLSLTTFGSSSSSNTTAVMSPGGTTSFLDVLRGGTGGLLDGNLGQNNGYYYGGSRSGIGMLMTPPAASFGIPGPMQQHGDLMVGGNGIGAATASIFQGGTGEEGDDGKGAMMGLQWQPHVGNGGGGGVVSGGVHHLGTGNNVTMGNNNINNNNNNGSHSDDNTGGSSRDCYWINNGGSNPWQSLLNSSSLM

SEQ ID NO: 7 - DNA, 2081 bp, Triticum aestivum
ctggcttgctcattctgcggtagtgtttaaacatcagctagccttacgggtataaaaaggtgggcaacttcaccctatcccatagcactagaccaaacaacacctatactccatactacccttcattcacctggtgagattcttcttcctttgatctctatcacttactctttctcccttcttcttcttcttcttcttcttcttcttcttcttcttcttcttcttcttcttcttcttcttcctcgtgcatgctacttttgctacataatctcctgcagtatcgctcgccgcaagctgtgctagctagctagcgatcttcactttaagacccgttgtagatctagccacgggctattccaaaaaatatttctcttgtttgcgtgttcctgtgtacatgcatgtatttagatcttgatcttgaagaattcatactgaattcatgacctaccttgtttggtttgtgtaattttgatgttgttgtatcaattttagcaaaccgctcgtagctagaacaatagagggggcggccgtatgtttccatttcgaaaaggggatatttccaggctctgcatcggttcatgcacacagccgttaccacattcaataggcactaatccatggatgcatgccagatttactagttttgtttacaaagttttatttttttttgctttgatttacgaaaaattggatcggattttgcagttcttttttatccatgttggattcactactttgaacccaagattttatttattttgtctcggtttcttacacgcctggttaggtaactaggagatcctgggattaggctttcaaggaatcctaatactagagagtatggggagaggcaccttattttttaagttgcccaaaagaatgggcgcttagagttgtagctaattaattgaatctgttgtatggatctgagaatttgagacctgattatgcacttatcatgacattttgagtcaaccaatgattctacatgtctcactccttaggattaacaatttaacttaatttaattcgatatgtgtgtacacatgtgttgtagatggaggaagtgtttccgtcaaactccaagagcaaggcaggtcagatggcgggggaggcgatagcgggggcggagaagaagcctcggccaaagccagagcagaaggtggaatgccctcggtgcaagtctggcaacaccaagttctgctactacaacaactatagtatgtctcagccccgctacttctgcaaggcctgccgccgctactggacccatggtggctccctccgcaacgtccccatcggtggtggctgccgcaagcccaagcgctcggggacctccgacgcccacaagctcggcgtggcctcctcaccggaacccacgactgtcgtgcccccctcgacctgcacagggatgaactttgcgaacgtcctcccgacgtttatgtctgttggttttgagattccaagcagcctttccctaaccgcctttgggtcatcatcgtcgtccaacacggcggcgatgatgtcccctggtgggacgacgtcatttctagacgtgctaagagggggtgcaggagggcttcttgatggcagcctcagtcagaacaatggctactactatggtgggccagccattggatcaggcaatgggatgctgatgacgccgccagcggtgtcatttggcattccagttccgatgcagcagcatggtgatctcgtggttggtggaaatggaataggtgccgcaactgcttcaatatttcaaggggccactagcgaggaaggagatgacggcatggggggcgtgatggggctccaatggcaaccacaggttggcaatggtggaggtggtggtggtgtatcaggaggcgtgcatcaccttgggactgggaacaatgtgacgatgggcaacagcaacatacacaacaacaacaataacgacagcggcggtgatgacaacaatggtgggtcatcgagggattgctactggatcaacaatggaggatcaaacccatggcagagcctcctcaacagcagctccctgatgtaagtgcaagaagaaaatgggaaatggaggtcat

SEQ ID NO: 8 - DNA, 990 bp, Triticum aestivum
atggaggaagtgtttccgtcaaactccaagagcaaggcaggtcagatggcgggggaggcgatagcgggggcggagaagaagcctcggccaaagccagagcagaaggtggaatgccctcggtgcaagtctggcaacaccaagttctgctactacaacaactatagtatgtctcagccccgctacttctgcaaggcctgccgccgctactggacccatggtggctccctccgcaacgtccccatcggtggtggctgccgcaagcccaagcgctcggggacctccgacgcccacaagctcggcgtggcctcctcaccggaacccacgactgtcgtgcccccctcgacctgcacagggatgaactttgcgaacgtcctcccgacgtttatgtctgttggttttgagattccaagcagcctttccctaaccgcctttgggtcatcatcgtcgtccaacacggcggcgatgatgtcccctggtgggacgacgtcatttctagacgtgctaagagggggtgcaggagggcttcttgatggcagcctcagtcagaacaatggctactactatggtgggccagccattggatcaggcaatgggatgctgatgacgccgccagcggtgtcatttggcattccagttccgatgcagcagcatggtgatctcgtggttggtggaaatggaataggtgccgcaactgcttcaatatttcaaggggccactagcgaggaaggagatgacggcatggggggcgtgatggggctccaatggcaaccacaggttggcaatggtggaggtggtggtggtgtatcaggaggcgtgcatcaccttgggactgggaacaatgtgacgatgggcaacagcaacatacacaacaacaacaataacgacagcggcggtgatgacaacaatggtgggtcatcgagggattgctactggatcaacaatggaggatcaaacccatggcagagcctcctcaacagcagctccctgatg

SEQ ID NO: 9 - protein, 330 aa, Triticum aestivum
MEEVFPSNSKSKAGQMAGEAIAGAEKKPRPKPEQKVECPRCKSGNTKFCYYNNYSMSQPRYFCKACRRYWTHGGSLRNVPIGGGCRKPKRSGTSDAHKLGVASSPEPTTVVPPSTCTGMNFANVLPTFMSVGFEIPSSLSLTAFGSSSSSNTAAMMSPGGTTSFLDVLRGGAGGLLDGSLSQNNGYYYGGPAIGSGNGMLMTPPAVSFGIPVPMQQHGDLVVGGNGIGAATASIFQGATSEEGDDGMGGVMGLQWQPQVGNGGGGGGVSGGVHHLGTGNNVTMGNSNIHNNNNNDSGGDDNNGGSSRDCYWINNGGSNPWQSLLNSSSLM

SEQ ID NO: 10 - DNA, 189 bp, Triticum aestivum
aagccagagcagaaggtggaatgccctcggtgcaagtctggcaacaccaagttctgctactacaacaactatagtatgtctcagccccgctacttctgcaaggcctgccgccgctactggacccatggtggctccctccgcaacgtccccatcggtggtggctgccgcaagcccaagcgctcggggacc

SEQ ID NO: 11 - protein, 63 aa, Triticum aestivum
KPEQKVECPRCKSGNTKFCYYNNYSMSQPRYFCKACRRYWTHGGSLRNVPIGGGCRKPKRSGT

SEQ ID NO: 12 - protein, 284 aa, Hordeum vulgare
MEEVFSSNSKSKAGQMAGEAAAAAEKKSRPKPEQKVECPRCKSGNTKFCYYNNYSMSQPRYFCKACRRYWTHGGSLRNVPIGGGCRKPKRPGTSDAHKLGMASSSEPTGVVPPSNCTGMNFANVLPTFMSGGFDIQSSLSLTTFGSSSSSNPTALMSPGGTTSFLDVLRGGAGGLLDGSLGPNNGYYYGGHANGSSIGMLMTPPTVSFGIPSPMQQHGGLVVGGNGIGGTTSSTFQGSAGEEGDDGTGSIMGLQWQPHVGNGGGGVVGLGGAHHLGTGNNVTMG

(19) United States
(12) Patent Application Publication
(10) Pub. No.: US 2023/0413751 A1
(43) Pub. Date: Dec. 28, 2023

(54) METHODS OF INCREASING FIBER IN A WHEAT GRAIN

(71) Applicant: ARCADIA BIOSCIENCES, INC., Davis, CA (US)

(72) Inventors: Jos VAN BOXTEL, Davis, CA (US); Charles Paul MOEHS, Davis, CA (US); William J. AUSTILL, Davis, CA (US); Dayna LOEFFLER, Davis, CA (US); Jessica MULLENBERG, Davis, CA (US)

(21) Appl. No.: 18/025,135

(22) PCT Filed: Sep. 7, 2021
(86) PCT No.: PCT/US21/49202

§ 371 Date: Mar. 7, 2023

Related U.S. Application Data
(60) Provisional application No. 63/075,278, filed on Sep. 7, 2020.

(51) Int. Cl. A01H 1/00; A01H 6/46; A01H 5/10

(52) U.S. Cl. CPC A01H 1/103; A01H 6/4678; A01H 5/10

(57) ABSTRACT

The present application relates to methods for wheat with increased dietary fiber. Methods of increasing dietary fiber by providing mutations in Wheat Prolamin-box Binding Factor are disclosed.

This application claims the priority benefit of U.S. Provisional Patent Application Ser. No. 63/075,278, filed Sep. 7, 2020, which is hereby incorporated by reference in its entirety.

This invention was made with government support under DK097976 awarded by the National Institutes of Health. The government has certain rights in the invention.

### FIELD

Disclosed herein are methods and compositions for providing wheat with increased fiber content.

### BACKGROUND

Wheat is an important and strategic cereal crop and is the most important staple food of about two billion people (36% of the world population). Worldwide, wheat provides nearly 55% of the carbohydrates and 20% of the food calories consumed. Wheat exceeds in acreage and production every other grain crop (including rice, maize, etc.) and is cultivated over a wide range of climatic conditions.

The wheat genome is five times larger than the human genome and forty times larger than the rice genome. In addition, bread wheat (Triticum aestivum) is a hexaploid, with three complete genomes termed A, B and D in the nucleus of each cell. Durum wheat (Triticum durum or Triticum turgidum subsp. durum), is the major tetraploid species of wheat of commercial importance, which is widely cultivated today. Durum wheat has two complete genomes, A and B, and is widely used for making pasta.

A current problem in many areas of the world is the increasing prevalence of adults and children that are either overweight or obese. According to the FDA, almost 40% of adults in the United States are obese, and 70% are overweight or obese. Increasing fiber in the diet is often recommended to help promote weight loss, reduce obesity, reduce the risk of coronary heart disease and colon cancers and to improve overall health. Some of the physiological effects of dietary fiber include lowering blood pressure, cholesterol, and blood glucose levels. Fiber also helps to promote a feeling of fullness and to improve bowel health with increased laxation and mineral absorption. Sources of dietary fiber include grains such as wheat.

There is a need to increase consumption of fiber to combat obesity, decrease the prevalence of other types of disease, and provide laxation and other benefits attributed to fiber. Because it is prevalent in many consumers' diets, increasing the fiber content of wheat would help increase overall fiber consumption. Although the need has been long felt, the identification of methods to increase the fiber content of wheat has proceeded slowly because, among other possible reasons, there is limited genetic diversity in today's commercial wheat cultivars, the wheat genome is complex, and few methods to increase fiber in wheat are known.

The present application is directed to overcoming these and other deficiencies in the art.

### BRIEF DESCRIPTION OF THE DRAWINGS

FIG. 1 shows a protein sequence alignment of the Wheat Prolamin-box Binding Factor (“WPBF”) of wheat (WPBF_A (SEQ ID NO:3), WPBF_D (SEQ ID NO:6), and WPBF_B (SEQ ID NO:9)) and barley (SEQ ID NO:12). The DNA binding with one finger (“DoF”) domain is boxed and highly conserved residues are indicated in bold font.

FIG. 2 is a photographic image of wheat grains from WPBF high fiber mutant lines (WPBF20A and 20B), compared to grains from wild-type controls (WPBF21), and grains from conventional Northern Plains or Express varieties.

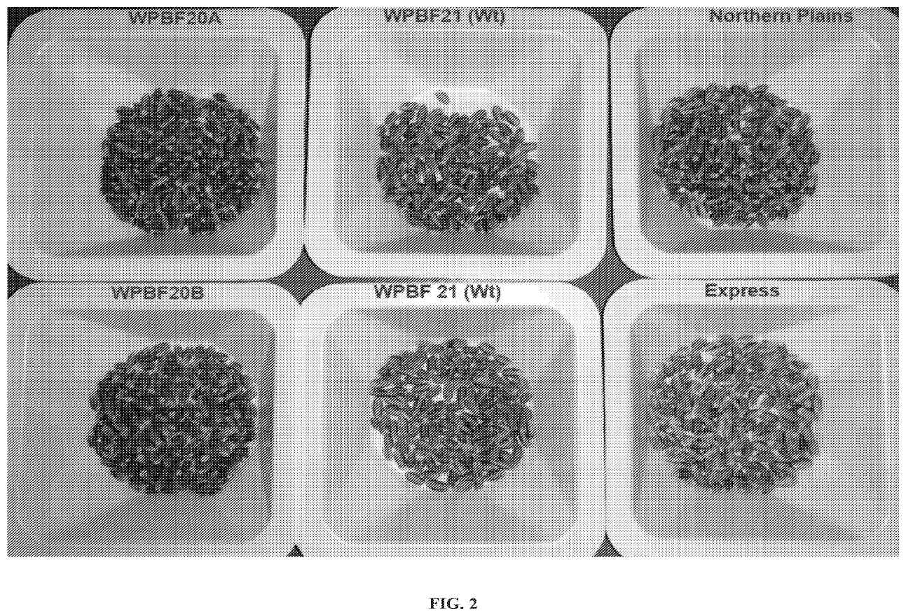

### SUMMARY

One aspect of the present application relates to a method of increasing fiber in a wheat grain. This method involves providing a wheat plant or plant part comprising a Wheat Prolamin-box Binding Factor (WPBF) gene and introducing a human-induced mutation into the WPBF gene, where the mutation is effective to create a wheat plant capable of producing wheat grain with increased fiber compared to wheat grain from a wheat plant not having the mutation.

Another aspect of the present application relates to a method of producing dietary fiber. This method involves providing a wheat plant comprising a human-induced mutation in a Wheat Prolamin-box Binding Factor (WPBF) gene, where the mutation causes the wheat plant to produce grain with increased fiber compared to a wheat plant without the mutation and obtaining fiber from the grain of the wheat plant.

A further aspect of the present application relates to a wheat plant capable of producing grain with elevated fiber content, the wheat plant comprising a human-induced mutation in a Wheat Prolamin-box Binding Factor (WPBF) gene, where the mutation is effective to elevate fiber in the grain of a wheat plant compared to grain from a wheat plant not having the mutation.

Another aspect of the present application relates to a method of selecting wheat grain with high fiber content. This method involves introducing a human-induced mutation into a wheat plant or plant part, producing a wheat plant having the mutation, where the wheat plant produces grain. The method further involves identifying grain from the produced wheat plant having an embryo larger than an embryo of wheat grain from a wild-type wheat plant, and selecting the grain with the larger embryo as wheat grain with higher fiber content.

The inventors have discovered methods of increasing the dietary fiber component of wheat by mutating and modifying genes never previously thought or known to be involved in the production of grain with increased dietary fiber. It is surprisingly shown herein that mutations in the WPBF gene increase the fiber content in wheat grain and flour. Unexpectedly, one fiber component that was increased in wheat grain of mutated plants was identified as resistant starch, a form of starch that is not readily digested into glucose, but instead persists to the lower digestive tract where it is fermented.

### DETAILED DESCRIPTION

A first aspect of the present application is directed to a method of increasing fiber in a wheat grain. This method involves providing a wheat plant or plant part comprising a Wheat Prolamin-box Binding Factor (WPBF) gene and introducing a human-induced mutation into the WPBF gene, where the mutation is effective to create a wheat plant capable of producing wheat grain with increased fiber compared to wheat grain from a wheat plant not having the mutation.

As used herein, “fiber” includes non-digestible soluble and insoluble carbohydrates with 3 or more monomeric units. In wheat, fiber is abundant in the outer layers of the grain, known as the bran, and includes arabinoxylans and cellulose. In addition to the bran, arabinoxylans are also found in the cell walls of the starchy endosperm. Fructans are another component of dietary fiber in wheat, present at around 1%-2.5% of the grain. Fructans are rapidly digested, short oligosaccharide polymers that contain fructose. Beta-glucan, a polysaccharide of D-glucose monomers linked by β-glycosidic bonds, accounts for <1% of the fiber in wheat. A minor component (<1%) of starch in conventional varieties is resistant to digestion and is considered a desirable dietary fiber called resistant starch.

Wheat Prolamin-box Binding Factor (WPBF), a DNA binding with one finger (DoF) transcription factor, functions as an activator of prolamin gene expression during seed development. WPBF is an activator of storage protein gene expression. During central endosperm development, the transcription of the genes encoding storage proteins is temporally and spatially regulated through a pathway that requires transcription factors that bind to specific DNA motifs, including the endosperm box (“EB”) and the ACAA motif. The EB has two distinct protein binding sites: the GCN4-like motif and the prolamin box.

The Dof proteins are plant transcription factors that have a highly conserved DNA-binding domain. The Dof domain, which is composed of about 50-60 amino acid residues, is similar to the Cys2/Cys2 zinc finger DNA-binding domain of GATA1 and steroid hormone receptors, but has a longer putative loop as compared to zinc-finger domains.

A recently discovered mutation in the barley prolamin box binding factor was shown to be responsible for the high lysine content and low hordein (seed storage protein) phenotype in the mutant, lys3a (Moehs et al., “Development of Decreased-Gluten Wheat Enabled by Determination of the Genetic Basis of lys3a Barley,” Plant Phys. 179:1692-1703 (2019), which is hereby incorporated by reference in its entirety). Other barley lines with mutations in LYS3 were recently sequenced (Orman-Ligeza et al., “LYS3 encodes a Prolamin-Box-Binding Transcription Factor that controls Embryo Growth in Barley and Wheat,” Journal of Cereal Science 93:102965 (2020), which is hereby incorporated by reference in its entirety). One of the barley lys3 mutants, M1460 (lys3d), was originally selected based on a reduced beta-glucan phenotype (Aastrup, “Selection and Characterization of Low 13-Glucan Mutants from Barley,” Carlsberg Res. Commun. 48:307-316 (1983), which is hereby incorporated by reference in its entirety).

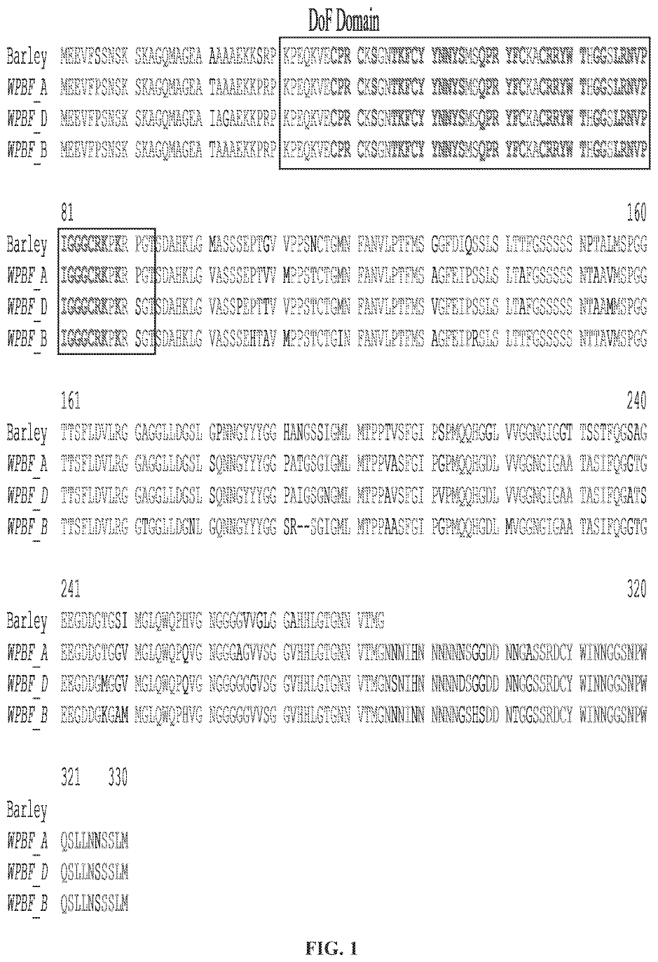

Wheat WPBF genes encode a protein similar to barley LYS3 (SEQ ID NO:12) as shown in the alignment of FIG. 1. In bread wheat, grains with combined mutations in all three WPBF genes (WPBF_A (SEQ ID NO:1), WPBF_B (SEQ ID NO:4), and WPBF_D (SEQ ID NO:7)) have been reported to have reduced seed storage proteins such as gliadins and low molecular weight glutenins, and also have large embryos (see Moehs et al., “Development of Decreased-Gluten Wheat Enabled by Determination of the Genetic Basis of lys3a Barley,” Plant Phys. 179:1692-1703 (2019); Orman-Ligeza et al., “LYS3 encodes a Prolamin-Box-Binding Transcription Factor that controls Embryo Growth in Barley and Wheat,” Journal of Cereal Science 93:102965 (2020), which are hereby incorporated by reference in their entirety). However, none of these reports associate mutating WPBF as a method to increase dietary fiber content of a wheat grain.

As used herein, the terms “increasing,” “increased,” “reduced,” “inhibits” or the like are considered relative terms, i.e. in comparison with the wild-type or unaltered state. The “level” of a protein refers to the amount of a particular protein, for example WPBF, which may be measured by any means known in the art such as, for example, Western blot analysis, other immunological means, or mass spectrometry. As used herein, “transcription factor (“TF”) activity” refers to the extent to which the TF activates the transcription of its target genes.

As used herein, “WPBF activity” may be measured by one or more of the following characteristics: (1) the extent to which WPBF activates transcription; (2) the extent to which WPBF binds DNA; (3) the extent to which WPBF binds to co-activators and/or other transcriptional regulatory complexes; and (4) the stability of WPBF bound to DNA. It would be appreciated that the level of WPBF activity or the level of transcription factor activity might be altered in a mutant but not the expression level (amount) of the protein itself. Conversely, the amount of protein might be altered but the activity remain the same if a more or less active protein is produced.

Reductions in both level of protein and protein activity are also possible such as, for example, when a gene encoding the enzyme is inactivated. In certain embodiments, the reduction in the level of protein or activity is by at least 10% or by at least 20% or by at least 30% or by at least 40% or by at least 50% or by at least 60% compared to the level of protein or activity in the endosperm of unmodified wheat, or by at least 70%, or by at least 80% or by at least 85% or by at least 90% or at least 95%. In certain embodiments, the reduction in the level of protein or activity is 100%. In some embodiments, the level of protein or protein activity is undetectable. The reduction in the level of the protein or gene expression or level of WPBF activity or level of transcription factor activity may occur at any stage in the development of the grain, particularly during the grain filling stage, or at all stages of grain development through to maturity.

As used herein, the term “allele” is any of one or more alternative forms of a gene, all of which relate to one trait or characteristic. In a diploid cell or organism, the two alleles of a given gene occupy corresponding loci on a pair of homologous chromosomes. In a tetraploid or hexaploid cell or organism, such as wheat, the two alleles of a given gene on one of the genomes occupy corresponding loci on a pair of homologous chromosomes and the two alleles of the same gene occupying the same loci on another of the genomes such as the A or B genomes of tetraploid, or the A, B, or D genomes of hexaploid wheat are said to be homoeologous to the gene of the first genome and to be present on homoeologous chromosomes.

As used herein, amino acid or nucleotide sequence identity and similarity are determined from an optimal global alignment between the two sequences being compared. An optimal global alignment is achieved using, for example, the Needleman-Wunsch algorithm (Needleman and Wunsch, “A General Method Applicable to the Search for Similarities in the Amino Acid Sequence of Two Proteins,” J. Mol. Biol. 48:443-453 (1970), which is hereby incorporated by reference in its entirety). Sequences may also be aligned using algorithms known in the art including but not limited to CLUSTAL V algorithm or the BLASTN or BLAST 2 sequence programs.

As used herein, “identity” means that an amino acid or nucleotide at a particular position in a first polypeptide or polynucleotide is identical to a corresponding amino acid or nucleotide in a second polypeptide or polynucleotide that is in an optimal global alignment with the first polypeptide or polynucleotide. In contrast to identity, “similarity” encompasses amino acids that are conservative substitutions. A “conservative” substitution is any substitution that has a positive score in the Blosum62 substitution matrix (Henikoff and Henikoff, “Amino Acid Substitution Matrices from Protein Blocks,” Proc. Natl. Acad. Sci. USA 89:10915-10919 (1992), hereby incorporated by reference in its entirety).

As used herein, the term “plant” means an immature or mature whole plant, including a plant from which seed or grain or anthers have been removed. A seed or embryo that will produce a plant is also considered to be a plant.

As used herein, the term “plant part(s)” includes plant protoplasts, plant cell tissue cultures from which wheat plants can be regenerated, plant calli, plant clumps, and plant cells that are intact in plants or parts of plants, such as embryos, pollen, ovules, pericarp, seed, flowers, florets, heads, spikes, leaves, roots, root tips, anthers, and the like.

As used herein, the term “polypeptide(s)” refers to any peptide or protein comprising two or more amino acids joined to each other by peptide bonds or modified peptide bonds. Polypeptide(s) refers to both short chains, commonly referred to as peptides, oligopeptides, and oligomers, and to longer chains generally referred to as proteins. Polypeptides may contain amino acids other than the 20 gene-encoded amino acids. Polypeptide(s) include those modified either by natural processes, such as processing and other post-translational modifications, but also by chemical modification techniques. Such modifications are well described in basic texts and in more detailed monographs, as well as in a voluminous research literature and they are well known to those of skill in the art. It will be appreciated that the same type of modification may be present in the same or varying degree at several sites in a given polypeptide.

As used herein, the term “polynucleotide(s)” or “nucleotide(s)” generally refers to any polyribonucleotide or poly-deoxyribonucleotide, which may be unmodified RNA or DNA or modified RNA or DNA. This definition includes, without limitation, single- and double-stranded DNA, DNA that is a mixture of single- and double-stranded regions or single-, double- and triple-stranded regions, cDNA, single- and double-stranded RNA, and RNA that is a mixture of single- and double-stranded regions, hybrid molecules comprising DNA and RNA that may be single-stranded or, more typically, double-stranded, or triple-stranded regions, or a mixture of single- and double-stranded regions. The term polynucleotide(s) also embraces short nucleotides or fragments, often referred to as oligonucleotides, that due to mutagenesis are not 100% identical but nevertheless code for the same amino acid sequence.

The term “reduced function” refers to a nucleic acid sequence that encodes a WPBF protein that has reduced biological activity as compared to the protein coding sequence of the whole nucleic acid sequence. In other words, it refers to a nucleic acid or fragment(s) thereof that substantially retains the capacity of encoding a WPBF polypeptide of the present application, but the encoded WPBF polypeptide has reduced activity.

The term “fragment,” as used herein, refers to a polynucleotide sequence (e.g, a PCR fragment) which is an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase, or any other polymerizing technique well known in the art, or expressed in a host cell by recombinant nucleic acid technology well known to one of skill in the art.

As used herein, a single nucleotide polymorphism (“SNP”) is a single nucleotide base difference between two DNA according to nucleotide substitutions either as transitions (C/T or G/A) or transversions (C/G, A/T, C/A or T/G). Additional types of mutations include insertions, deletions, translocation, and any combination thereof. Insertions and deletions can range from more than 1 to thousands of bases.

As used herein, a “transgenic” plant refers to a plant that contains a gene construct (“transgene”) not found in a wild-type plant of the same species, variety or cultivar. A transgene has the normal meaning in the art of biotechnology and includes a genetic sequence that has been produced or altered by recombinant DNA or RNA technology and which has been introduced into the plant cell. The transgene may include genetic sequences derived from a plant cell. Typically, the transgene has been introduced into the plant by human manipulation such as, for example, by transformation but any method can be used as one of skill in the art recognizes.

As used herein, a “non-transgenic” or “modified” plant refers to a plant that has a non-transgenic mutation, or a plant that has undergone genomic editing or combinations thereof. As used herein, a “modified WPBF gene” includes modification of the WPBF gene through non-transgenic mutations or transgenes or genomic editing or combinations thereof. A “human-induced” mutation refers to a mutation that has been introduced, either through chemical mutagenesis or genome modification.

A wheat plant is defined herein as any plant of a species of the genus Triticum, which species is commercially cultivated, including, for example, Triticum aestivum L. ssp. aestivum (common or bread wheat), other subspecies of Triticum aestivum, Triticum turgidum L. ssp. durum (durum wheat, also known as macaroni or pasta wheat), Triticum monococcum L. ssp. monococcum (cultivated einkorn or small spelt), Triticum timopheevi ssp. timopheevi, Triticum turgidum L. ssp. dicoccon (cultivated emmer), and other subspecies of Triticum turgidum (Feldman). The wheat may be hexaploid wheat having an AABBDD type genome, or tetraploid wheat having an AABB type genome. Since genetic variation in wheat transferred to certain related species, including rye and barley by hybridization, the present application also includes the hybrid species thus formed, including triticale that is a hybrid between bread wheat and rye. In one embodiment, the wheat plant is of the species Triticum aestivum, and preferably of the subspecies aestivum. In another embodiment the wheat plant is of the species Triticum turgidum L. ssp. durum. Additionally, mutations or transgenes can be readily transferred from Triticum aestivum to durum wheat.

As used herein, the term “barley” refers to any species of the Genus Hordeum, including progenitors thereof as well as progeny thereof produced by crosses with other species. A preferred form of barley is the species Hordeum vulgare. Barley LYS3 amino acid sequence is shown below (GenBank Accession No. MN715387, which is hereby incorporated by reference in its entirety) SEQ ID NO:12:

MEEVFSSNSK SKAGQMAGEA AAAAEKKSRP KPEQKVECPR
CKSGNTKFCY YNNYSMSQPR YFCKACRRYW THGGSLRNVP
IGGGCRKPKR PGTSDAHKLG MASSSEPTGV VPPSNCTGMN
FANVLPTFMS GGFDIQSSLS LTTFGSSSSS NPTALMSPGG
TTSFLDVLRG GAGGLLDGSL GPNNGYYYGG HANGSSIGML
MTPPTVSFGI PSPMQQHGGL VVGGNGIGGT TSSTFQGSAG
EEGDDGTGSI MGLQWQPHVG NGGGGVVGLG GAHHLGTGNN
VTMG

The present application describes wheat plants exhibiting grains with increased fiber as compared to wild type wheat plants without the inclusion of foreign nucleic acids in the wheat plant genome. In one embodiment, the present application relates to non-transgenic human-induced mutations in one or more WPBF genes.

In still another embodiment, the present application relates to introducing a series of independent human-induced mutations in one or more WPBF genes; wheat plants having one or more of these mutations in at least one WPBF gene thereof; and a method of creating and identifying similar and/or additional mutations in at least one WPBF gene of wheat to obtain wheat grain with increased amounts of fiber.

In yet another embodiment, the present application relates to a transgenic wheat plant with a transgene that reduces expression of the WPBF gene and/or activity of the WPBF protein, wherein said transgene contributes to grain having increased fiber as compared to grain from a wild type plant.

In still another embodiment, the present application relates to a wheat plant having a modified WPBF gene, where the WPBF gene is modified by genomic editing, and further, where said modification contributes to grain having increased fiber as compared to grain from a wild type plant.

In one embodiment, the present application relates to modifying the WPBF gene through human-induced mutations, transgenes, or genomic editing.

In one embodiment, the present application relates to one or more human-induced mutations in the WPBF gene. In one embodiment, the present application relates to multiple human-induced mutations in the WPBF gene including but not limited to 1, 2, 3, 4, 5, 6, 7, 8, 9, 10, and greater than 10 mutations.

In another embodiment, the WPBF gene may contain one or more human-induced mutations recited in Tables 1-3 and corresponding mutations in homoeologues and combinations thereof.

In another embodiment, the present application relates to corresponding mutations to the one or more human-induced mutations disclosed herein in the WPBF gene in a corresponding homoeologue. By way of example, an identified mutation in the WPBF gene of the A genome may also be a beneficial mutation in the WPBF gene of the B and/or D genome. One of ordinary skill in the art will understand that the mutation in the homoeologue may not be in the exact same location.

In some embodiments, the present application relates to a method where the mutation is introduced into the WPBF gene of the A genome, the B genome, or the D genome. In some embodiments, the present application relates to a method where the mutation is introduced into each of the WPBF genes of the A and B genomes. In some embodiments, the present application relates to a method where the mutation is introduced into each of the WPBF genes of the A and D genomes. In some embodiments, the present application relates to a method where the mutation is introduced into each of the WPBF genes of the B and D genomes. In some embodiments, the present application relates to a method where the mutation is introduced into each of the WPBF genes of the A, B, and D genomes.

One of ordinary skill in the art understands that there may be natural variation in the genetic sequences of the WPBF genes in different wheat varieties.

The inventors have determined that to achieve increased fiber in grains from plants, mutations that reduce WPBF gene function are desirable. In some embodiments the mutation is a loss of function mutation. The phrase “loss of function mutation” refers to a inactivating mutations that typically result in the gene product having less or no function compared to a wild-type gene product. Loss of function mutations include mutations that prematurely truncate the translation of one or more WPBF proteins from messenger RNA, such as those mutations that create a stop codon (nonsense mutations) within the coding region of a WPBF messenger RNA. Loss of function mutations also include splice junctions that throw the coding sequence out of reading frame, and insertions and deletions that alter the reading frame. Other mutations that do not change the reading frame (such as a missense mutation), but alter highly conserved amino acids in the DoF domain are considered loss of function mutations. The most highly conserved residues of the DoF domain are shown in FIG. 1 in bold font.

### 1. A Genome

In one embodiment, the present application relates to a human-induced mutation in the WPBF gene of the A genome including but not limited to 1, 2, 3, 4, 5, 6, 7, 8, 9, 10, and greater than 10 mutations. In one embodiment, one or more human-induced mutations are in both alleles of the WPBF gene in the A genome. In another embodiment, the human-induced mutations are identical in both alleles of the WPBF gene of the A genome. In one embodiment, the mutations are homozygous.

The following mutations identified in Tables 1-3 are exemplary of the mutations created and identified according to various embodiments disclosed herein. They are offered by way of illustration, not limitation. It is to be understood that the mutations below are merely exemplary and that similar mutations are also contemplated.

SEQ ID NO. 1: Wheat Prolamin-box Binding Factor (WPBF) A genome (the
nucleotide sequence may differ very slightly in different wheat
cultivars-different alleles):
CTGGCTTGCT CATTTTGCGG TAGTGTTTAA ACATCGGCTA GCCTTACGGG TATAAAAAGG
TGGGCAACTT CACCCTATCC CATAGCACTA GACCAAAGAA CACCTATACT CCATACTACC
CTTCGTTCAC CTGGTGAGCT TCTTCTTCCT TTGATCTATA TCACTTACTA TTTCTCCCTT
GTCCAGCTTC TTCTTCTTCC TCGTGCATGC GACTTTTTCT AGATAATATC CCGCACTATC
GCTCGCCGCA AGATGTGCTA GCTAGCGATC TTCACTTTAA TACCTGTTGT AGATCTAACC
ACGGGCTATT CCAAAAAATA TTTGTCTTGT TTGCGTGTTC CTGTGTACAT GCACGTATCT
AGATCTTGAT TTTGAAGAAT TCATAATTAA TTCATGACCT ACCTTGTTTG GTTTGTGTAA
TTTTGATGTT GTCGTATCAA TTTTAGCAAA CCACTCGTAG CTAGAACAAT AGAGGGGGCG
ATCGTATGTT TCTGTTTTGA AAAGGGGATA TTTCCAGGCT CTGCATCGGT TCATGCACAC
AGCCGTTACC ACATTCAATA GGCACTGATC CATGGATGCA TGCCAGATTT ACTAGTTTTG
TATACAAAGT TTTACTTTTT TGCTTTGATT TATGAAAAGT TGGATCAGAT TTTGCAGTTC
TCTTTTATCC ATGTTGGATT CACTACTTTG TACCCAAGAT TTTATTTATT TTGTCTTGGT
TTCTTACCTG CCTGGTTAGT AACTAGGAGA TCCTGGGATT AGACTTTCAA GGAATCCTAA
TACTAGTGAG TATAGGGAAA GGAAGCTTAT TTTTAAGCTG CCCAAAAGAA TGGGCGCTTA
GAGTTGTAGT TGATTAATTG AATCTGTTCT GTGGATTTGA GAATTTCAGA CCTGATTCTA
CATGACATTT TGAGTTAACC AATGATTCTA CATGTCTCAC TCCTTGGGAT TAACAATTTA
ACTTTATTTA ATTCGATATG TGTGTACACA TGTGTTGCAG ATGGAGGAAG TGTTTCCGTC
AAACTCCAAG AGCAAGGCCG GTCAGATGGC GGGGGAGGCG ACAGCGGCGG CGGAGAAGAA
GCCTCGGCCG AAGCCAGAGC AGAAGGTGGA ATGCCCTCGG TGCAAGTCTG GCAACACCAA
GTTCTGCTAC TACAACAACT ATAGTATGTC TCAGCCCCGC TACTTCTGCA AGGCCTGCCG
CCGCTACTGG ACCCATGGTG GCTCCCTCCG CAACGTCCCC ATCGGTGGCG GCTGCCGCAA
GCCCAAGCGC CCGGGGACCT CCGACGCCCA CAAGCTCGGC GTGGCCTCCT CGTCAGAACC
CACGGTTGTC ATGCCGCCCT CGACCTGCAC AGGGATGAAC TTTGCCAACG TCCTCCCAAC
ATTTATGTCT GCTGGTTTTG AGATTCCAAG CAGCCTTTCC CTGACTGCCT TTGGGTCATC
GTCATCGTCC AACACGGCGG CAGTGATGTC CCCTGGTGGG ACGACGTCAT TTCTAGACGT
GTTGAGAGGG GGCGCAGGAG GGCTTCTTGA TGGCAGCCTC AGTCAGAACA ATGGCTACTA
CTATGGTGGG CCTGCCACTG GATCAGGCAT TGGGATGCTG ATGACGCCGC CAGTGGCGTC
ATTTGGCATT CCAGGTCCGA TGCAGCAACA TGGTGATCTC GTGGTTGGTG GAAATGGAAT
AGGTGCTGCA ACTGCTTCAA TATTTCAGGG GGGCACTGGC GAGGAAGGAG ATGATGGTAC
GGGGGGCGTG ATGGGGCTCC AATGGCAGCC ACAGGTTGGC AATGGTGGAG GTGCTGGTGT
TGTATCAGGA GGCGTGCATC ACCTTGGGAC TGGGAACAAT GTGACGATGG GCAACAACAA
TATACACAAC AACAACAATA ACAACAGTGG GGGTGATGAC AACAATGGTG CGTCATCGAG
GGATTGCTAC TGGATCAACA ATGGAGGATC GAACCCATGG CAGAGCCTCC TCAACAACAG
CTCCCTGATG TAAGTGCAAT AAGAAAATGG GAAATGGAGG TCAT
SEQ ID NO: 2 Wheat Prolamin-box Binding Factor (WPBF) A genome
Coding Region:
ATGGAGGAAG TGTTTCCGTC AAACTCCAAG AGCAAGGCCG GTCAGATGGC GGGGGAGGCG
ACAGCGGCGG CGGAGAAGAA GCCTCGGCCG AAGCCAGAGC AGAAGGTGGA ATGCCCTCGG
TGCAAGTCTG GCAACACCAA GTTCTGCTAC TACAACAACT ATAGTATGTC TCAGCCCCGC
TACTTCTGCA AGGCCTGCCG CCGCTACTGG ACCCATGGTG GCTCCCTCCG CAACGTCCCC
ATCGGTGGCG GCTGCCGCAA GCCCAAGCGC CCGGGGACCT CCGACGCCCA CAAGCTCGGC
GTGGCCTCCT CGTCAGAACC CACGGTTGTC ATGCCGCCCT CGACCTGCAC AGGGATGAAC
TTTGCCAACG TCCTCCCAAC ATTTATGTCT GCTGGTTTTG AGATTCCAAG CAGCCTTTCC
CTGACTGCCT TTGGGTCATC GTCATCGTCC AACACGGCGG CAGTGATGTC CCCTGGTGGG
ACGACGTCAT TTCTAGACGT GTTGAGAGGG GGCGCAGGAG GGCTTCTTGA TGGCAGCCTC
AGTCAGAACA ATGGCTACTA CTATGGTGGG CCTGCCACTG GATCAGGCAT TGGGATGCTG
ATGACGCCGC CAGTGGCGTC ATTTGGCATT CCAGGTCCGA TGCAGCAACA TGGTGATCTC
GTGGTTGGTG GAAATGGAAT AGGTGCTGCA ACTGCTTCAA TATTTCAGGG GGGCACTGGC
GAGGAAGGAG ATGATGGTAC GGGGGGCGTG ATGGGGCTCC AATGGCAGCC ACAGGTTGGC
AATGGTGGAG GTGCTGGTGT TGTATCAGGA GGCGTGCATC ACCTTGGGAC TGGGAACAAT
GTGACGATGG GCAACAACAA TATACACAAC AACAACAATA ACAACAGTGG GGGTGATGAC
AACAATGGTG CGTCATCGAG GGATTGCTAC TGGATCAACA ATGGAGGATC GAACCCATGG
CAGAGCCTCC TCAACAACAG CTCCCTGATG
SEQ ID NO. 3: WPBF-A genome amino acid sequence:
MEEVFPSNSK SKAGQMAGEA TAAAEKKPRP KPEQKVECPR CKSGNTKFCY YNNYSMSQPR
YFCKACRRYW THGGSLRNVP IGGGCRKPKR PGTSDAHKLG VASSSEPTVV MPPSTCTGMN
FANVLPTFMS AGFEIPSSLS LTAFGSSSSS NTAAVMSPGG TTSFLDVLRG GAGGLLDGSL
SQNNGYYYGG PATGSGIGML MTPPVASFGI PGPMQQHGDL VVGGNGIGAA TASIFQGGTG
EEGDDGTGGV MGLQWQPQVG NGGGAGVVSG GVHHLGTGNN VTMGNNNIHN NNNNNSGGDD
NNGASSRDCY WINNGGSNPW QSLLNNSSLM

Table 1 provides a list of representative mutations in the WPBF gene in the A genome. One exemplary mutation is G41A, resulting in a change from guanine to adenine at nucleotide position 41 identified according to its position in the sequence of SEQ ID NO: 2. This mutation results in a change from glycine to aspartic acid at amino acid position 14 identified according to its position in the expressed protein (SEQ ID NO: 3).

TABLE 1
Representative Mutations in the WPBF Gene in the A Genome

| Wheat | Gene | Mutation | Mutation Score | Nucleotide |
|---|---|---|---|---|
| Kronos | WPBF-A | G14D | Missense | G41A |
| Kronos | WPBF-A | E19K | Missense | G55A |
| Kronos | WPBF-A | A22T | Missense | G64A |
| Kronos | WPBF-A | A23T | Missense | G67A |
| Kronos | WPBF-A | P28S | Severe Missense | C82T |
| Kronos | WPBF-A | P32S | Missense | C94T |
| Kronos | WPBF-A | E33K | Missense | G97A |
| Kronos | WPBF-A | C49Y | Severe Missense | G146A |
| Kronos | WPBF-A | W70* | Nonsense | G210A |
| Kronos | WPBF-A | G74D | Severe Missense | G221A |
| Kronos | WPBF-A | L76F | Severe Missense | C226T |
| Kronos | WPBF-A | G84S | Severe Missense | G250A |
| Kronos | WPBF-A | G84D | Severe Missense | G251A |
| Kronos | WPBF-A | A96V | Missense | C287T |
| Kronos | WPBF-A | E106K | Missense | G316A |
| Kronos | WPBF-A | V124I | Missense | G370A |
| Kronos | WPBF-A | P126S | Missense | C376T |
| Kronos | WPBF-A | A143V | Missense | C428T |
| Kronos | WPBF-A | A143V | Missense | C428T |
| Kronos | WPBF-A | A153T | Missense | G457A |
| Kronos | WPBF-A | G171D | Severe Missense | G512A |
| Kronos | WPBF-A | G174R | Severe Missense | G520A |
| Kronos | WPBF-A | G178D | Severe Missense | G533A |
| Express | WPBF-A | G18R | Missense | G52A |
| Express | WPBF-A | V36M | Missense | G106A |
| Express | WPBF-A | C66Y | Severe Missense | G197A |
| Express | WPBF-A | P80L | Severe Missense | C239T |
| Express | WPBF-A | G92R | Missense | G274A |
| Express | WPBF-A | S103F | Severe Missense | C308T |
| Express | WPBF-A | V109I | Missense | G325A |
| Express | WPBF-A | G145E | Severe Missense | G434A |
| Express | WPBF-A | A131V | Missense | C392T |
| Express | WPBF-A | S138N | Missense | G413A |
| Express | WPBF-A | S146L | Missense | C437T |
| Express | WPBF-A | S150F | Missense | C449T |
| Express | WPBF-A | T152M | Missense | C445T |
| Express | WPBF-A | A153T | Missense | G457A |
| Express | WPBF-A | A154T | Missense | G460A |
| Express | WPBF-A | S157F | Missense | C470T |
| Express | WPBF-A | P158S | Missense | C472T |
| Express | WPBF-A | G160E | Missense | G479A |
| Express | WPBF-A | T162M | Missense | C485T |
| Express | WPBF-A | S163L | Missense | C488T |
| Express | WPBF-A | D166G | Missense | A497G |
| Express | WPBF-A | G170E | Missense | G509A |
| Express | WPBF-A | G171D | Missense | G512A |
| Express | WPBF-A | A172T | Missense | G514A |
| Express | WPBF-A | G172R | Missense | G520A |
| Express | WPBF-A | G178D | Missense | G533A |
| Express | WPBF-A | G185S | Missense | G553A |
| Express | WPBF-A | G185D | Missense | G554A |
| Express | WPBF-A | G189D | Missense | G566A |
| Express | WPBF-A | G190E | Missense | G569A |
| Express | WPBF-A | P191L | Missense | C575T |
| Express | WPBF-A | G194E | Missense | C576T |
| Express | WPBF-A | G196D | Missense | G581A |
| Express | WPBF-A | M199I | Missense | G1587A |
| Express | WPBF-A | A206T | Missense | G594A |
| Express | WPBF-A | S207L | Missense | G597A |
| Express | WPBF-A | G209D | Missense | G626A |
| Express | WPBF-A | P211S | Missense | C631T |
| Express | WPBF-A | P211L | Missense | C632T |
| Express | WPBF-A | G212D | Missense | G635A |
| Express | WPBF-A | M214I | Missense | G642A |
| Express | WPBF-A | Q215* | Nonsense | C643T |
| Express | WPBF-A | H217Y | Missense | C649T |
| Express | WPBF-A | G281D | Missense | G653A |
| Express | WPBF-A | G223D | Severe Missense | G668A |
| Express | WPBF-A | G224R | Severe Missense | G670A |
| Express | WPBF-A | G224E | Severe Missense | G671A |
| Express | WPBF-A | G226E | Severe Missense | G677A |
| Express | WPBF-A | G228D | Severe Missense | G683A |
| Express | WPBF-A | A229T | Severe Missense | G685A |
| Express | WPBF-A | A230V | Severe Missense | C689T |
| Express | WPBF-A | A232V | Severe Missense | C695T |
| Express | WPBF-A | Q236L | Severe Missense | A707T |
| Express | WPBF-A | G237R | Severe Missense | G709A |
| Express | WPBF-A | G237E | Severe Missense | G710A |
| Express | WPBF-A | G238S | Severe Missense | G712A |
| Express | WPBF-A | G238D | Severe Missense | G713A |
| Express | WPBF-A | T239I | Severe Missense | C716T |
| Express | WPBF-A | G240D | Severe Missense | G719A |
| Express | WPBF-A | E241K | Severe Missense | G721A |
| Express | WPBF-A | E242K | Severe Missense | G724A |
| Express | WPBF-A | G243E | Severe Missense | G728A |
| Express | WPBF-A | D244N | Missense | G730A |
| Express | WPBF-A | G246D | Severe Missense | G737A |
| Express | WPBF-A | G248R | Severe Missense | G742A |
| Express | WPBF-A | G249D | Missense | G746A |
| Express | WPBF-A | M251I | Missense | G753A |
| Express | WPBF-A | G252R | Missense | G754A |
| Express | WPBF-A | Q254* | Nonsense | C760T |
| Express | WPBF-A | W255* | Nonsense | G765A |
| Express | WPBF-A | G262D | Missense | G785A |
| Express | WPBF-A | G264S | Missense | G790A |
| Express | WPBF-A | A265T | Missense | G793A |
| Express | WPBF-A | A265V | Missense | C794T |
| Express | WPBF-A | S269L | Missense | C806T |
| Express | WPBF-A | G270E | Missense | G809A |
| Express | WPBF-A | G271S | Missense | G811A |
| Express | WPBF-A | G271D | Missense | G812A |
| Express | WPBF-A | L275F | Missense | C823T |
| Express | WPBF-A | G276R | Missense | G826A |
| Express | WPBF-A | G276E | Missense | G827A |
| Express | WPBF-A | G278E | Missense | G833A |
| Express | WPBF-A | V281M | Missense | G841A |
| Express | WPBF-A | T282M | Missense | C845T |
| Express | WPBF-A | G284S | Missense | G850A |
| Express | WPBF-A | G284D | Missense | G851A |
| Express | WPBF-A | G297R | Severe Missense | G889A |
| Express | WPBF-A | G297E | Severe Missense | G890A |
| Express | WPBF-A | G298S | Missense | G892A |
| Express | WPBF-A | G298D | Missense | G893A |
| Express | WPBF-A | D299N | Missense | G895A |
| Express | WPBF-A | D300N | Missense | G898A |
| Express | WPBF-A | A304V | Missense | C911T |
| Express | WPBF-A | S305L | Missense | C914T |
| Express | WPBF-A | S306L | Severe Missense | C917T |
| Express | WPBF-A | G315E | Severe Missense | G944A |
| Express | WPBF-A | S317L | Severe Missense | C950T |
| Express | WPBF-A | P319S | Missense | C955T |
| Express | WPBF-A | W320* | Nonsense | G960A |
| Express | WPBF-A | Q321* | Nonsense | C961T |
| Express | WPBF-A | L323F | Severe Missense | C967T |
| Express | WPBF-A | S327N | Missense | G980A |
| Express | WPBF-A | S328F | Missense | C983T |
| Kronos | WPBF-A | M1I | Severe Missense | G3A |
| Kronos | WPBF-A | E3K | Missense | G7A |
| Kronos | WPBF-A | P6S | Missense | C16T |
| Kronos | WPBF-A | G14S | Missense | G40A |
| Kronos | WPBF-A | A17T | Missense | G49A |
| Kronos | WPBF-A | E19K | Missense | G55A |
| Kronos | WPBF-A | A22T | Missense | G64A |
| Kronos | WPBF-A | A23T | Missense | G67A |
| Kronos | WPBF-A | A24T | Missense | G70A |
| Kronos | WPBF-A | P30S | Missense | C88T |
| Kronos | WPBF-A | Q34L | Missense | A101T |
| Kronos | WPBF-A | V36M | Missense | G106A |
| Kronos | WPBF-A | E37K | Missense | G109A |
| Kronos | WPBF-A | P39S | Severe Missense | C115T |
| Kronos | WPBF-A | G44D | Missense | G131A |
| Kronos | WPBF-A | T46I | Severe Missense | C137T |
| Kronos | WPBF-A | C49Y | Severe Missense | G146A |
| Kronos | WPBF-A | M56I | Severe Missense | G168A |
| Kronos | WPBF-A | R60L | Severe Missense | G179T |
| Kronos | WPBF-A | G73D | Severe Missense | G218A |
| Kronos | WPBF-A | S75F | Severe Missense | C224T |
| Kronos | WPBF-A | P80S | Severe Missense | C238T |
| Kronos | WPBF-A | P80L | Severe Missense | C239T |
| Kronos | WPBF-A | G84D | Severe Missense | G251A |
| Kronos | WPBF-A | P88L | Severe Missense | C263T |
| Kronos | WPBF-A | P91S | Missense | C271T |
| Kronos | WPBF-A | A96V | Missense | C287T |
| Kronos | WPBF-A | L99F | Missense | C295T |
| Kronos | WPBF-A | A102V | Missense | C305T |
| Kronos | WPBF-A | S103F | Missense | C308T |
| Kronos | WPBF-A | E106K | Missense | G316A |
| Kronos | WPBF-A | P113S | Missense | C337T |
| Kronos | WPBF-A | P113L | Missense | C338T |
| Kronos | WPBF-A | T115I | Missense | C344T |
| Kronos | WPBF-A | I124V | Missense | A370G |
| Kronos | WPBF-A | L125F | Missense | C373T |
| Kronos | WPBF-A | P126S | Missense | C376T |
| Kronos | WPBF-A | S140F | Missense | C419T |
| Kronos | WPBF-A | G171D | Missense | G512A |

In one embodiment, the present application relates to a polynucleotide of the WPBF gene in the A genome with one or more human-induced mutations listed in Table 1 and corresponding to SEQ ID NO: 2. In another embodiment, the polynucleotide with one or more human-induced mutations listed in Table 1 has 85%, 86%, 87%, 88%, 89%, 90%, 91%, 92%, 93%, 94%, 95%, 96%, 97%, 98%, 99% or greater than 99% identity to SEQ ID NO: 2.

In still another embodiment, the polynucleotide with one or more human-induced mutation listed in Table 1 codes for a WPBF protein, wherein the WPBF protein comprises one or more human-induced mutations and has 85%, 86%, 87%, 88%, 89%, 90%, 91%, 92%, 93%, 94%, 95%, 96%, 97%, 98%, 99% or greater than 99% identity to SEQ ID NO: 3.

### 2. B Genome

In one embodiment, the present application relates to a human-induced mutation in the WPBF gene of the B genome including but not limited to 1, 2, 3, 4, 5, 6, 7, 8, 9, 10, and greater than 10 mutations. In one embodiment, one or more human-induced mutations are in both alleles of the WPBF gene in the B genome. In another embodiment, the human-induced mutations are identical in both alleles of the WPBF gene of the B genome. In still another embodiment, the mutations are homozygous.

SEQ ID NO: 4 WPBF-B genome:
CTGGCTTGCT CATTTTGCGG TAGTGTTTAA ACATTGGCTG GAATTACGGG TATAAAAAGG
AGGGCAACTT CACCCTATCC CATAGCACTA GACCAAACAA CTCCTATACT CCATACTACC
CTTCATTCAC CTGGTGAGCT TCTTCTTTCT TTGATTTCTA TCACTTACTC TTTCTCCCTC
GTCCAGCTTC TTCTTCTTCC TCGTGCATGT GACTTTTGCT AGATAATCTC CCACATTATC
GCTCAATGCA AGCCGTGCTA GCTAGCTAGC GATCTAGCTA GCGATCTTCA CTTTAATACC
CGTTGTAGAT CTAACCATGG GCTATTCCAA AACATATTTC TCTTGTTTGC GTGTTCGTGT
GTACATGCAT GCATCTAGAT CTTGATTTTG AGGAATTCAT AAGTAATTCC TGACCTACCT
TGTTTGGTTT GTTTAATTTT GATGTTGTTG TCTCAATTTT AGCAAATTGC TCGTAGCTAG
AACAATAGAG GGGGCGGCCG TATGTTTCCG TTTTGAAAAG GGGATATTTC CAGGCTCTGC
ATCGGTTGAT GCACACAGCC GTTACCACAT TCAATAGGCA CTGATCCATG GATGCATGCT
ATATTTACAA GTTTTCTATA GAAATTTTTT TTATTTATGA AAAATTGGAT CGGTATAGTT
CTTCTTTATC CATGTCGGAT TCACTACTTT GTACCCAAGA TTTTATTTAT TTTGTCTCGG
TTTCTTACAT GTCTAGTTAG GTAACTAGGA GAGCCTGGGA TTAGGCTTTC AAGGAATCCT
AATACTAGAG ACTATGGGGA GAGACAGCTT ATTCTTTAAG CTGCGCAAAA GAATGGGCGC
TTAGAGTTGT AGTTGATAAA TTGAATCTGT TGTATGGATT TGAGAATTTG AGACCTGATT
ATGCACTTAT CATGAAATTT TGAGTTAACC AATGATTCTA CATGTCTCAC TCCTTAGGAT
TAACAATTTA ACTTAATTTA ATTCGATATG TGTGTACACA TGTGTTGAAG ATGGAGGAAG
TGTTTCCGTC AAACTCCAAG AGCAAGGCTG GTCAGATGGC GGGGGAGGCG ACAGCGGCGG
CGGAGAAGAA GCCTCGGCCG AAGCCAGAGC AGAAGGTGGA ATGCCCTCGG TGCAAGTCTG
GCAACACCAA GTTCTGCTAC TACAACAACT ATAGTATGTC TCAGCCCCGC TACTTCTGCA
AGGCCTGCCG CCGCTACTGG ACCCATGGTG GGTCCCTCCG TAACGTCCCC ATCGGTGGTG
GCTGCCGCAA GCCCAAGCGC TCGGGGACCT CCGACGCCCA CAAGCTCGGC GTGGCCTCCT
CGTCGGAACA CACGGCTGTC ATGCCCCCCT CGACCTGCAC AGGGATAAAC TTTGCCAATG
TCCTCCCGAC GTTTATGTCT GCTGGTTTTG AGATTCCAAG AAGCCTTTCC CTGACCACCT
TTGGGTCATC GTCGTCGTCC AACACGACGG CTGTCATGTC CCCTGGTGGG ACGACGTCAT
TTCTAGACGT GCTGAGAGGG GGAACAGGAG GGCTTCTTGA TGGCAACCTC GGTCAGAACA
ATGGCTACTA CTATGGTGGG TCTAGATCAG GCATTGGGAT GCTGATGACG CCGCCAGCGG
CGTCATTTGG CATTCCAGGT CCAATGCAGC AGCATGGCGA TCTCATGGTT GGTGGAAATG
GAATAGGTGC CGCAACTGCT TCAATATTTC AGGGGGGCAC TGGTGAGGAA GGAGATGACG
GCAAAGGGGC CATGATGGGG CTCCAATGGC AGCCACATGT TGGTAATGGT GGAGGTGGTG
GTGTTGTATC AGGAGGCGTG CATCACCTTG GGACTGGGAA CAATGTGACG ATGGGCAACA
ACAACATAAA CAACAATAAC AATAATGGCA GCCACAGTGA TGACAACACT GGTGGGTCAT
CGAGGGATTG CTACTGGATC AATAATGGAG GATCGAACCC ATGGCAAAGC CTCCTCAATA
GCAGCTCCCT GATGTAAGTG CAAGAAGAAA ATGCGAAATG GAGATCAT
SEQ ID NO. 5: WPBF-B genome coding sequence:
ATGGAGGAAG TGTTTCCGTC AAACTCCAAG AGCAAGGCTG GTCAGATGGC GGGGGAGGCG
ACAGCGGCGG CGGAGAAGAA GCCTCGGCCG AAGCCAGAGC AGAAGGTGGA ATGCCCTCGG
TGCAAGTCTG GCAACACCAA GTTCTGCTAC TACAACAACT ATAGTATGTC TCAGCCCCGC
TACTTCTGCA AGGCCTGCCG CCGCTACTGG ACCCATGGTG GGTCCCTCCG TAACGTCCCC
ATCGGTGGTG GCTGCCGCAA GCCCAAGCGC TCGGGGACCT CCGACGCCCA CAAGCTCGGC
GTGGCCTCCT CGTCGGAACA CACGGCTGTC ATGCCCCCCT CGACCTGCAC AGGGATAAAC
TTTGCCAATG TCCTCCCGAC GTTTATGTCT GCTGGTTTTG AGATTCCAAG AAGCCTTTCC
CTGACCACCT TTGGGTCATC GTCGTCGTCC AACACGACGG CTGTCATGTC CCCTGGTGGG
ACGACGTCAT TTCTAGACGT GCTGAGAGGG GGAACAGGAG GGCTTCTTGA TGGCAACCTC
GGTCAGAACA ATGGCTACTA CTATGGTGGG TCTAGATCAG GCATTGGGAT GCTGATGACG
CCGCCAGCGG CGTCATTTGG CATTCCAGGT CCAATGCAGC AGCATGGCGA TCTCATGGTT
GGTGGAAATG GAATAGGTGC CGCAACTGCT TCAATATTTC AGGGGGGCAC TGGTGAGGAA
GGAGATGACG GCAAAGGGGC CATGATGGGG CTCCAATGGC AGCCACATGT TGGTAATGGT
GGAGGTGGTG GTGTTGTATC AGGAGGCGTG CATCACCTTG GGACTGGGAA CAATGTGACG
ATGGGCAACA ACAACATAAA CAACAATAAC AATAATGGCA GCCACAGTGA TGACAACACT
GGTGGGTCAT CGAGGGATTG CTACTGGATC AATAATGGAG GATCGAACCC ATGGCAAAGC
CTCCTCAATA GCAGCTCCCT GATG
SEQ ID NO. 6 WPBF-B genome amino acid sequence:
MEEVFPSNSK SKAGQMAGEA TAAAEKKPRP KPEQKVECPR CKSGNTKFCY YNNYSMSQPR
YFCKACRRYW THGGSLRNVP IGGGCRKPKR SGTSDAHKLG VASSSEHTAV MPPSTCTGIN
FANVLPTFMS AGFEIPRSLS LTTFGSSSSS NTTAVMSPGG TTSFLDVLRG GTGGLLDGNL
GQNNGYYYGG SRSGIGMLMT PPAASFGIPG PMQQHGDLMV GGNGIGAATA SIFQGGTGEE
GDDGKGAMMG LQWQPHVGNG GGGGVVSGGV HHLGTGNNVT MGNNNINNNN NNGSHSDDNT
GGSSRDCYWI NNGGSNPWQS LLNSSSLM

Table 2 provides a representative list of mutations in the WPBF gene of the B genome, of wheat plants, Kronos and Express. Nucleotide and amino acid changes are identified according to their positions in SEQ ID NOs: 5 and 6, respectively. The “*” indicates a stop codon.

TABLE 2
Representative Mutations in the WPBF_Gene of the B Genome

| Wheat | Gene | Mutation | Mutation Score | Nucleotide |
|---|---|---|---|---|
| Kronos | WPBF-B | V4E | Missense | TllA |
| Kronos | WPBF-B | S9F | Missense | C26T |
| Kronos | WPBF-B | A13T | Missense | G37A |
| Kronos | WPBF-B | Ml61 | Missense | G48A |
| Kronos | WPBF-B | Al7T | Missense | G49A |
| Kronos | WPBF-B | Gl8R | Missense | G52A |
| Kronos | WPBF-B | El9K | Missense | G55A |
| Kronos | WPBF-B | A20T | Missense | G58A |
| Kronos | WPBF-B | A22T | Missense | G64A |
| Kronos | WPBF-B | A23T | Missense | G67A |
| Kronos | WPBF-B | A24T | Missense | G70A |
| Kronos | WPBF-B | A24V | Missense | C71T |
| Kronos | WPBF-B | P28L | Severe Missense | C83T |
| Kronos | WPBF-B | P30S | Missense | C88T |
| Kronos | WPBF-B | P32L | Missense | C95T |
| Kronos | WPBF-B | E37K | Missense | G109A |
| Kronos | WPBF-B | C38Y | Missense | Gll3A |
| Kronos | WPBF-B | P39S | Severe Missense | C115T |
| Kronos | WPBF-B | S43F | Severe Missense | Cl28T |
| Kronos | WPBF-B | T461 | Severe Missense | Cl37T |
| Kronos | WPBF-B | Q58* | Nonsense | Cl72T |
| Kronos | WPBF-B | P59S | Severe Missense | Cl75T |
| Kronos | WPBF-B | T711 | Severe Missense | C212T |
| Kronos | WPBF-B | G73S | Severe Missense | G217A |
| Kronos | WPBF-B | G73D | Severe Missense | G218A |
| Kronos | WPBF-B | G74R | Severe Missense | G221A |
| Kronos | WPBF-B | R77H | Severe Missense | G230A |
| Kronos | WPBF-B | P80S | Severe Missense | C238T |
| Kronos | WPBF-B | P80L | Severe Missense | C239T |
| Kronos | WPBF-B | G83S | Severe Missense | G247A |
| Kronos | WPBF-B | P88L | Severe Missense | C263T |
| Kronos | WPBF-B | G92E | Severe Missense | G275A |
| Kronos | WPBF-B | S94F | Missense | C281T |
| Kronos | WPBF-B | A96V | Missense | C287T |
| Kronos | WPBF-B | Al02T | Missense | G304A |
| Kronos | WPBF-B | Al02V | Missense | C305T |
| Kronos | WPBF-B | Sl05L | Missense | C314T |
| Kronos | WPBF-B | El06K | Missense | G316A |
| Kronos | WPBF-B | Al09T | Missense | G325A |
| Kronos | WPBF-B | Al09V | Missense | C326T |
| Kronos | WPBF-B | Mllll | Missense | G333A |
| Kronos | WPBF-B | G118E | Missense | G353A |
| Kronos | WPBF-B | Al22V | Missense | C365T |
| Kronos | WPBF-B | L125F | Missense | C373T |
| Kronos | WPBF-B | Pl26S | Missense | C376T |
| Kronos | WPBF-B | Pl26L | Missense | C377T |
| Kronos | WPBF-B | Ml291 | Missense | G387A |
| Kronos | WPBF-B | Pl36S | Missense | C406T |
| Kronos | WPBF-B | Pl36L | Missense | C407T |
| Kronos | WPBF-B | L139F | Missense | C415T |
| Kronos | WPBF-B | Sl40F | Missense | C419T |
| Kronos | WPBF-B | T1421 | Missense | C425T |
| Kronos | WPBF-B | T1431 | Missense | C428T |
| Kronos | WPBF-B | Gl45E | Missense | G434A |
| Kronos | WPBF-B | Sl47L | Missense | C440T |
| Kronos | WPBF-B | Sl50F | Missense | C449T |
| Kronos | WPBF-B | Ml561 | Missense | G468A |
| Kronos | WPBF-B | Gl59D | Missense | G476A |
| Kronos | WPBF-B | Gl60E | Missense | G479A |
| Kronos | WPBF-B | Sl63F | Missense | C487T |
| Kronos | WPBF-B | S163L | Missense | C488T |
| Kronos | WPBF-B | Rl69K | Missense | G506A |
| Kronos | WPBF-B | Gl71R | Missense | G511A |
| Kronos | WPBF-B | Gl74E | Missense | G521A |
| Kronos | WPBF-B | L176F | Missense | C526T |
| Express | WPBF-B | E2K | Missense | G4A |
| Express | WPBF-B | E3K | Missense | G7A |
| Express | WPBF-B | E3D | Missense | A9T |
| Express | WPBF-B | A13T | Missense | G37A |
| Express | WPBF-B | Gl4D | Missense | G41A |
| Express | WPBF-B | Ml61 | Missense | G48A |
| Express | WPBF-B | Al 7T | Missense | G49A |
| Express | WPBF-B | Gl8R | Missense | G52A |
| Express | WPBF-B | Gl8E | Missense | G53A |
| Express | WPBF-B | A20T | Missense | G58A |
| Express | WPBF-B | A22T | Missense | G64A |
| Express | WPBF-B | A22V | Missense | G65A |
| Express | WPBF-B | A23T | Missense | G67A |
| Express | WPBF-B | A24T | Missense | G70A |
| Express | WPBF-B | P28L | Severe Missense | C83T |
| Express | WPBF-B | P32T | Severe Missense | C94A |
| Express | WPBF-B | P32S | Severe Missense | C94T |
| Express | WPBF-B | E37K | Missense | G109A |
| Express | WPBF-B | C38Y | Severe Missense | Gll3A |
| Express | WPBF-B | P39L | Severe Missense | C116T |
| Express | WPBF-B | R40W | Missense | C118T |
| Express | WPBF-B | G44S | Missense | G130A |
| Express | WPBF-B | G44D | Missense | G131A |
| Express | WPBF-B | T461 | Severe Missense | Cl37T |
| Express | WPBF-B | Q58* | Nonsense | Cl72T |
| Express | WPBF-B | P59S | Severe Missense | Cl75T |
| Express | WPBF-B | P59L | Missense | Cl76T |
| Express | WPBF-B | R60C | Severe Missense | Cl78T |
| Express | WPBF-B | R60H | Severe Missense | G179A |
| Express | WPBF-B | C63Y | Severe Missense | G188A |
| Express | WPBF-B | A65V | Missense | Cl94T |
| Express | WPBF-B | R68H | Severe Missense | G203A |
| Express | WPBF-B | W70* | Nonsense | G209A |
| Express | WPBF-B | W70R | Severe Missense | T208A |
| Express | WPBF-B | T711 | Severe Missense | C212T |
| Express | WPBF-B | G73D | Severe Missense | G218A |
| Express | WPBF-B | G73C | Missense | G217T |
| Express | WPBF-B | G73S | Missense | G217A |
| Express | WPBF-B | S75F | Severe Missense | C224T |
| Express | WPBF-B | L76F | Severe Missense | C226T |
| Express | WPBF-B | PS0S | Severe Missense | C238T |
| Express | WPBF-B | PS0L | Severe Missense | C239T |
| Express | WPBF-B | G82D | Severe Missense | G245A |
| Express | WPBF-B | G83D | Severe Missense | G248A |
| Express | WPBF-B | G84S | Severe Missense | G250A |
| Express | WPBF-B | G84D | Severe Missense | G251A |
| Express | WPBF-B | P88L | Missense | C263T |
| Express | WPBF-B | R90H | Missense | G269A |
| Express | WPBF-B | G92E | Severe Missense | G275A |
| Express | WPBF-B | A96T | Missense | G286A |
| Express | WPBF-B | A96V | Missense | C287T |
| Express | WPBF-B | Gl00D | Missense | G299A |
| Express | WPBF-B | Vl0lM | Missense | G301A |
| Express | WPBF-B | El06K | Missense | G316A |
| Express | WPBF-B | H107Y | Missense | C319T |
| Express | WPBF-B | Al09T | Missense | G325A |
| Express | WPBF-B | P113S | Missense | C337T |
| Express | WPBF-B | P113L | Missense | C338T |
| Express | WPBF-B | T1151 | Missense | C344T |
| Express | WPBF-B | G118E | Missense | G353A |
| Express | WPBF-B | Al22S | Missense | G364T |
| Express | WPBF-B | Vl241 | Missense | G370A |
| Express | WPBF-B | L125F | Missense | C373T |
| Express | WPBF-B | Pl26S | Missense | C376T |
| Express | WPBF-B | Pl26L | Missense | C377T |
| Express | WPBF-B | Al31T | Missense | G391A |
| Express | WPBF-B | G132S | Missense | G394A |
| Express | WPBF-B | G132D | Missense | G395A |
| Express | WPBF-B | Pl36S | Missense | C406T |
| Express | WPBF-B | G145E | Missense | G434A |
| Express | WPBF-B | Al54T | Missense | G460A |
| Express | WPBF-B | Ml561 | Missense | G468A |
| Express | WPBF-B | Pl58S | Missense | C472A |
| Express | WPBF-B | G159D | Missense | G476A |
| Express | WPBF-B | T161M | Severe Missense | C482T |
| Express | WPBF-B | D166N | Severe Missense | G496A |
| Express | WPBF-B | G170R | Missense | G508A |
| Express | WPBF-B | G171R | Missense | G511A |
| Express | WPBF-B | G171E | Missense | G512A |
| Express | WPBF-B | G174R | Missense | G520A |
| Express | WPBF-B | G178D | Missense | G533A |
| Express | WPBF-B | Ql82* | Nonsense | C544T |
| Express | WPBF-B | G185S | Missense | G553A |
| Express | WPBF-B | G185D | Missense | G554A |
| Express | WPBF-B | G189D | Missense | G566A |
| Express | WPBF-B | Gl90R | Severe Missense | G568A |

In one embodiment, the present application relates to a polynucleotide of the WPBF gene in the B genome with one or more human-induced mutations listed in Table 2 and corresponding to SEQ ID NO: 5. In another embodiment, the polynucleotide with one or more human-induced mutations listed in Table 2 has 85%, 86%, 87%, 88%, 89%, 90%, 91%, 92%, 93%, 94%, 95%, 96%, 97%, 98%, 99% or greater than 99% identity to SEQ ID NO: 5.

In still another embodiment, the polynucleotide with one or more human-induced mutation listed in Table 2 codes for a WPBF protein, wherein the WPBF protein comprises one or more human-induced mutations and has 85%, 86%, 87%, 88%, 89%, 90%, 91%, 92%, 93%, 94%, 95%, 96%, 97%, 98%, 99% or greater than 99% identity to SEQ ID NO: 6.

### 3. D Genome

In one embodiment, the present application relates to a human-induced mutation in the WPBF gene of the D genome including but not limited to 1, 2, 3, 4, 5, 6, 7, 8, 9, 10, and greater than 10 mutations. In one embodiment, one or more human-induced mutations are in both alleles of the WPBF gene in the D genome. In another embodiment, the human-induced mutations are identical in both alleles of the WPBF gene of the D genome.

SEQ ID NO: 7 WPBF-D genome:
CTGGCTTGCT CATTCTGCGG TAGTGTTTAA ACATCAGCTA GCCTTACGGG TATAAAAAGG
TGGGCAACTT CACCCTATCC CATAGCACTA GACCAAACAA CACCTATACT CCATACTACC
CTTCATTCAC CTGGTGAGAT TCTTCTTCCT TTGATCTCTA TCACTTACTC TTTCTCCCTT
CTTCTTCTTC TTCTTCTTCT TCTTCTTCTT CTTCTTCTTC TTCTTCTTCT TCTTCTTCTT
CCTCGTGCAT GCTACTTTTG CTACATAATC TCCTGCAGTA TCGCTCGCCG CAAGCTGTGC
TAGCTAGCTA GCGATCTTCA CTTTAAGACC CGTTGTAGAT CTAGCCACGG GCTATTCCAA
AAAATATTTC TCTTGTTTGC GTGTTCCTGT GTACATGCAT GTATTTAGAT CTTGATCTTG
AAGAATTCAT ACTGAATTCA TGACCTACCT TGTTTGGTTT GTGTAATTTT GATGTTGTTG
TATCAATTTT AGCAAACCGC TCGTAGCTAG AACAATAGAG GGGGCGGCCG TATGTTTCCA
TTTCGAAAAG GGGATATTTC CAGGCTCTGC ATCGGTTCAT GCACACAGCC GTTACCACAT
TCAATAGGCA CTAATCCATG GATGCATGCC AGATTTACTA GTTTTGTTTA CAAAGTTTTA
TTTTTTTTTG CTTTGATTTA CGAAAAATTG GATCGGATTT TGCAGTTCTT TTTTATCCAT
GTTGGATTCA CTACTTTGAA CCCAAGATTT TATTTATTTT GTCTCGGTTT CTTACACGCC
TGGTTAGGTA ACTAGGAGAT CCTGGGATTA GGCTTTCAAG GAATCCTAAT ACTAGAGAGT
ATGGGGAGAG GCACCTTATT TTTTAAGTTG CCCAAAAGAA TGGGCGCTTA GAGTTGTAGC
TAATTAATTG AATCTGTTGT ATGGATCTGA GAATTTGAGA CCTGATTATG CACTTATCAT
GACATTTTGA GTCAACCAAT GATTCTACAT GTCTCACTCC TTAGGATTAA CAATTTAACT
TAATTTAATT CGATATGTGT GTACACATGT GTTGTAGATG GAGGAAGTGT TTCCGTCAAA
CTCCAAGAGC AAGGCAGGTC AGATGGCGGG GGAGGCGATA GCGGGGGCGG AGAAGAAGCC
TCGGCCAAAG CCAGAGCAGA AGGTGGAATG CCCTCGGTGC AAGTCTGGCA ACACCAAGTT
CTGCTACTAC AACAACTATA GTATGTCTCA GCCCCGCTAC TTCTGCAAGG CCTGCCGCCG
CTACTGGACC CATGGTGGCT CCCTCCGCAA CGTCCCCATC GGTGGTGGCT GCCGCAAGCC
CAAGCGCTCG GGGACCTCCG ACGCCCACAA GCTCGGCGTG GCCTCCTCAC CGGAACCCAC
GACTGTCGTG CCCCCCTCGA CCTGCACAGG GATGAACTTT GCGAACGTCC TCCCGACGTT
TATGTCTGTT GGTTTTGAGA TTCCAAGCAG CCTTTCCCTA ACCGCCTTTG GGTCATCATC
GTCGTCCAAC ACGGCGGCGA TGATGTCCCC TGGTGGGACG ACGTCATTTC TAGACGTGCT
AAGAGGGGGT GCAGGAGGGC TTCTTGATGG CAGCCTCAGT CAGAACAATG GCTACTACTA
TGGTGGGCCA GCCATTGGAT CAGGCAATGG GATGCTGATG ACGCCGCCAG CGGTGTCATT
TGGCATTCCA GTTCCGATGC AGCAGCATGG TGATCTCGTG GTTGGTGGAA ATGGAATAGG
TGCCGCAACT GCTTCAATAT TTCAAGGGGC CACTAGCGAG GAAGGAGATG ACGGCATGGG
GGGCGTGATG GGGCTCCAAT GGCAACCACA GGTTGGCAAT GGTGGAGGTG GTGGTGGTGT
ATCAGGAGGC GTGCATCACC TTGGGACTGG GAACAATGTG ACGATGGGCA ACAGCAACAT
ACACAACAAC AACAATAACG ACAGCGGCGG TGATGACAAC AATGGTGGGT CATCGAGGGA
TTGCTACTGG ATCAACAATG GAGGATCAAA CCCATGGCAG AGCCTCCTCA ACAGCAGCTC
CCTGATGTAA GTGCAAGAAG AAAATGGGAA ATGGAGGTCA T
SEQ ID NO. 8: WPBF-D coding sequence:
ATGGAGGAAG TGTTTCCGTC AAACTCCAAG AGCAAGGCAG GTCAGATGGC GGGGGAGGCG
ATAGCGGGGG CGGAGAAGAA GCCTCGGCCA AAGCCAGAGC AGAAGGTGGA ATGCCCTCGG
TGCAAGTCTG GCAACACCAA GTTCTGCTAC TACAACAACT ATAGTATGTC TCAGCCCCGC
TACTTCTGCA AGGCCTGCCG CCGCTACTGG ACCCATGGTG GCTCCCTCCG CAACGTCCCC
ATCGGTGGTG GCTGCCGCAA GCCCAAGCGC TCGGGGACCT CCGACGCCCA CAAGCTCGGC
GTGGCCTCCT CACCGGAACC CACGACTGTC GTGCCCCCCT CGACCTGCAC AGGGATGAAC
TTTGCGAACG TCCTCCCGAC GTTTATGTCT GTTGGTTTTG AGATTCCAAG CAGCCTTTCC
CTAACCGCCT TTGGGTCATC ATCGTCGTCC AACACGGCGG CGATGATGTC CCCTGGTGGG
ACGACGTCAT TTCTAGACGT GCTAAGAGGG GGTGCAGGAG GGCTTCTTGA TGGCAGCCTC
AGTCAGAACA ATGGCTACTA CTATGGTGGG CCAGCCATTG GATCAGGCAA TGGGATGCTG
ATGACGCCGC CAGCGGTGTC ATTTGGCATT CCAGTTCCGA TGCAGCAGCA TGGTGATCTC
GTGGTTGGTG GAAATGGAAT AGGTGCCGCA ACTGCTTCAA TATTTCAAGG GGCCACTAGC
GAGGAAGGAG ATGACGGCAT GGGGGGCGTG ATGGGGCTCC AATGGCAACC ACAGGTTGGC
AATGGTGGAG GTGGTGGTGG TGTATCAGGA GGCGTGCATC ACCTTGGGAC TGGGAACAAT
GTGACGATGG GCAACAGCAA CATACACAAC AACAACAATA ACGACAGCGG CGGTGATGAC
AACAATGGTG GGTCATCGAG GGATTGCTAC TGGATCAACA ATGGAGGATC AAACCCATGG
CAGAGCCTCC TCAACAGCAG CTCCCTGATG
SEQ ID NO. 9: WPBF-D amino acid sequence
MEEVFPSNSK SKAGQMAGEA IAGAEKKPRP KPEQKVECPR CKSGNTKFCY YNNYSMSQPR
YFCKACRRYW THGGSLRNVP IGGGCRKPKR SGTSDAHKLG VASSPEPTTV VPPSTCTGMN
FANVLPTFMS VGFEIPSSLS LTAFGSSSSS NTAAMMSPGG TTSFLDVLRG GAGGLLDGSL
SQNNGYYYGG PAIGSGNGML MTPPAVSFGI PVPMQQHGDL VVGGNGIGAA TASIFQGATS
EEGDDGMGGV MGLQWQPQVG NGGGGGGVSG GVHHLGTGNN VTMGNSNIHN NNNNDSGGDD
NNGGSSRDCY WINNGGSNPW QSLLNSSSLM

Table 3 provides representative examples of mutations created and identified in the WPBF gene in the D genome of wheat plants, Express. Nucleotide and amino acid changes are identified according to their positions in SEQ ID NOs: 8 and 9, respectively.

TABLE 3
Representative list of Mutations
in the WPBF_Gene of the D Genome

| Wheat | Gene | Mutation | Mutation Score | Nucleotide |
|---|---|---|---|---|
| Express | WPBF-D | C63Y | Severe Missense | G188A |
| Express | WPBF-D | S75F | Severe Missense | C224T |
| Express | WPBF-D | G82D | Severe Missense | G245A |
| Express | WPBF-D | P158L | Severe Missense | C473T |
| Express | WPBF-D | G170R | Severe Missense | G508A |
| Express | WPBF-D | E2K | Severe Missense | G4A |
| Express | WPBF-D | E3K | Severe Missense | G7A |
| Express | WPBF-D | S7L | Missense | C20T |
| Express | WPBF-D | A13T | Missense | G37A |
| Express | WPBF-D | A13V | Missense | C38T |
| Express | WPBF-D | A17T | Missense | G49A |
| Express | WPBF-D | E19K | Missense | G55A |
| Express | WPBF-D | A20T | Missense | G58A |
| Express | WPBF-D | A22T | Missense | G64A |
| Express | WPBF-D | A24T | Missense | G70A |
| Express | WPBF-D | E106K | Missense | G316A |
| Express | WPBF-D | P28S | Severe Missense | C82T |
| Express | WPBF-D | E37K | Missense | G109A |
| Express | WPBF-D | C38Y | Severe Missense | G113A |
| Express | WPBF-D | P39S | Severe Missense | C115T |
| Express | WPBF-D | P39L | Severe Missense | C116T |
| Express | WPBF-D | C41S | Severe Missense | T121A |
| Express | WPBF-D | G44D | Missense | G131A |
| Express | WPBF-D | C49Y | Severe Missense | G146A |
| Express | WPBF-D | R60C | Severe Missense | C178T |
| Express | WPBF-D | A65T | Missense | G193A |
| Express | WPBF-D | A65V | Missense | C194T |
| Express | WPBF-D | W70* | Nonsense | G209A |
| Express | WPBF-D | W70* | Nonsense | G210A |
| Express | WPBF-D | M119I | Missense | G357A |
| Express | WPBF-D | G73D | Severe Missense | G218A |
| Express | WPBF-D | G74D | Severe Missense | G221A |
| Express | WPBF-D | S75F | Severe Missense | C224T |
| Express | WPBF-D | P80S | Severe Missense | C238T |
| Express | WPBF-D | P80L | Severe Missense | C239T |
| Express | WPBF-D | G83S | Severe Missense | G247A |
| Express | WPBF-D | P88L | Missense | C263T |
| Express | WPBF-D | G92E | Severe Missense | G275A |
| Express | WPBF-D | S94F | Severe Missense | C281T |
| Express | WPBF-D | A96V | Missense | C287T |
| Express | WPBF-D | V101M | Missense | G301A |
| Express | WPBF-D | A102V | Missense | C305T |
| Express | WPBF-D | P105S | Missense | C313T |
| Express | WPBF-D | E106K | Missense | G316A |
| Express | WPBF-D | P107S | Severe Missense | C319T |
| Express | WPBF-D | P107L | Severe Missense | C320T |
| Express | WPBF-D | V111M | Missense | G331A |
| Express | WPBF-D | P112S | Severe Missense | C334T |
| Express | WPBF-D | P113L | Severe Missense | C338T |
| Express | WPBF-D | T115I | Missense | C344T |
| Express | WPBF-D | G118E | Missense | G353A |
| Express | WPBF-D | M119I | Missense | G357A |
| Express | WPBF-D | A154T | Missense | G460A |
| Express | WPBF-D | A122V | Missense | C365T |
| Express | WPBF-D | M129I | Missense | G387A |
| Express | WPBF-D | G123D | Missense | G395A |
| Express | WPBF-D | P136S | Missense | C406T |
| Express | WPBF-D | P136L | Missense | C407T |
| Express | WPBF-D | A143V | Missense | C428T |
| Express | WPBF-D | G145R | Missense | G433A |
| Express | WPBF-D | S150F | Missense | C449T |
| Express | WPBF-D | A153T | Missense | G457A |
| Express | WPBF-D | A154T | Missense | G460A |
| Express | WPBF-D | S157F | Missense | C470T |
| Express | WPBF-D | P158S | Missense | C472T |
| Express | WPBF-D | V221M | Missense | G661A |
| Express | WPBF-D | G159D | Missense | G476A |
| Express | WPBF-D | T161P | Severe Missense | A481C |
| Express | WPBF-D | R169K | Missense | G506A |
| Express | WPBF-D | G171S | Missense | G511A |
| Express | WPBF-D | G171D | Missense | G512A |
| Express | WPBF-D | G174R | Missense | G520A |
| Express | WPBF-D | G174E | Missense | G521A |
| Express | WPBF-D | L175F | Missense | C523T |
| Express | WPBF-D | G185S | Missense | G553A |
| Express | WPBF-D | G189D | Severe Missense | G566A |
| Express | WPBF-D | P191S | Missense | C571T |
| Express | WPBF-D | A192V | Missense | C575T |
| Express | WPBF-D | G194E | Severe Missense | G581A |
| Express | WPBF-D | G196D | Missense | G587A |
| Express | WPBF-D | G198E | Severe Missense | G593A |
| Express | WPBF-D | P203S | Missense | C607T |
| Express | WPBF-D | G249D | Severe Missense | G746A |
| Express | WPBF-D | P204S | Missense | C610T |
| Express | WPBF-D | A205T | Missense | G613A |
| Express | WPBF-D | V206M | Missense | G616A |
| Express | WPBF-D | G209S | Missense | G625A |
| Express | WPBF-D | G209D | Missense | G626A |
| Express | WPBF-D | V212I | Missense | G634A |
| Express | WPBF-D | M214I | Missense | G642A |
| Express | WPBF-D | Q215* | Nonsense | C643T |
| Express | WPBF-D | G218D | Severe Missense | G653A |
| Express | WPBF-D | D219N | Missense | G655A |
| Express | WPBF-D | G223S | Severe Missense | G667A |
| Express | WPBF-D | G223D | Missense | G668A |
| Express | WPBF-D | G228S | Missense | G682A |
| Express | WPBF-D | G228D | Severe Missense | G683A |
| Express | WPBF-D | A232V | Missense | C695T |
| Express | WPBF-D | S233L | Severe Missense | C698T |
| Express | WPBF-D | G237E | Severe Missense | G710A |
| Express | WPBF-D | A238V | Missense | C713T |
| Express | WPBF-D | T239I | Missense | C716T |
| Express | WPBF-D | G243R | Severe Missense | G727A |
| Express | WPBF-D | G243E | Severe Missense | G728A |
| Express | WPBF-D | D244N | Severe Missense | G730A |
| Express | WPBF-D | D245N | Severe Missense | G733A |
| Express | WPBF-D | M247I | Severe Missense | G741A |
| Express | WPBF-D | G248R | Severe Missense | G742A |
| Express | WPBF-D | G248E | Severe Missense | G743A |
| Express | WPBF-D | G249D | Severe Missense | G746A |
| Express | WPBF-D | G264S | Missense | G790A |
| Express | WPBF-D | G264D | Missense | G791A |
| Express | WPBF-D | G265D | Missense | G794A |
| Express | WPBF-D | G266D | Missense | G797A |
| Express | WPBF-D | G267D | Missense | G800A |
| Express | WPBF-D | G270E | Missense | G809A |
| Express | WPBF-D | G276R | Missense | G826A |
| Express | WPBF-D | G278E | Missense | G833A |
| Express | WPBF-D | G304R | Missense | G910A |

In one embodiment, the present application relates to a polynucleotide of the WPBF gene in the D genome with one or more human-induced mutations listed in Table 3 and corresponding to SEQ ID NO: 8. In another embodiment, the polynucleotide with one or more human-induced mutations listed in Table 3 and has 85%, 86%, 87%, 88%, 89%, 90%, 91%, 92%, 93%, 94%, 95%, 96%, 97%, 98%, 99% or greater than 99% identity to SEQ ID NO: 8.

In still another embodiment, the polynucleotide with one or more human-induced mutation listed in Table 3 codes for a WPBF protein, wherein the WPBF protein comprises one or more human-induced mutations and has 85%, 86%, 87%, 88%, 89%, 90%, 91%, 92%, 93%, 94%, 95%, 96%, 97%, 98%, 99% or greater than 99% identity to SEQ ID NO: 9.

### 4. Dof Region

In one embodiment, the present application relates to multiple human-induced mutations in the Dof region of WPBF gene of the A, B, or D genome. In one embodiment, the present application relates to multiple human-induced mutations in the Dof region of WPBF gene of the A and B genome. In one embodiment, the present application relates to multiple human-induced mutations in the Dof region of WPBF gene of the A and D genome. In one embodiment, the present application relates to multiple human-induced mutations in the Dof region of WPBF gene of the B and D genome.

In yet another embodiment, the present application relates to one or more human-induced mutations in the Dof region as shown in SEQ ID NO. 10 of the WPBF gene.

In yet another embodiment, the present application relates to one or more human-induced mutations in the Dof region as shown in SEQ ID NO. 11 of the WPBF protein. One or more of the 63 amino acids shown in SEQ ID NO. 11 can be mutated.

Dof Region of WPBF Gene
SEQ ID NO: 10
AAGCCAGAGC AGAAGGTGGA ATGCCCTCGG TGCAAGTCTG
GCAACACCAA GTTCTGCTAC TACAACAACT ATAGTATGTC
TCAGCCCCGC TACTTCTGCA AGGCCTGCCG CCGCTACTGG
ACCCATGGTG GCTCCCTCCG CAACGTCCCC ATCGGTGGTG
GCTGCCGCAA GCCCAAGCGC TCGGGGACC
Dof region of WPBF Protein
SEQ ID NO: 11
KPEQKVECPR CKSGNTKFCY YNNYSMSQPR YFCKACRRYW
THGGSLRNVP IGGGCRKPKR SGT

### WPBF Proteins

The present application relates to one or more human-induced mutations in the WPBF gene that result in a WPBF protein with one or more mutations as compared to wild type WPBF protein. In one embodiment, the human-induced mutations include but are not limited to the mutations recited in Tables 1-3, corresponding mutations in homoeologues, and combinations thereof.

In another embodiment, the present application relates to one or more human-induced mutations in the WPBF gene that inhibits production of the WPBF protein. In some embodiments, a mutation in the WPBF gene reduces expression of the WPBF protein. In other embodiments, a mutation in the WPBF gene creates an unstable or a WPBF protein with reduced function.

### 1. Expression Level of WPBF Protein

In another embodiment, the expression level of WPBF protein with one or more mutations disclosed herein is reduced by 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, or 95-99% of the expression level of the wild type WPBF protein.

In still another embodiment, the expression level of WPBF protein with one or more mutations disclosed herein is reduced from 10-20%, or from 20-30%, or from 30-40%, or from 40-50%, or from 50-60%, or from 60-70%, or from 70-80%, or from 80-90%, or from 90-99% as compared to the expression level of the wild type WPBF protein.

### 2. Activity of WPBF Protein

In yet another embodiment, the activity of the WPBF protein with one or more mutations disclosed herein is reduced to 0-1, 1, 2, 3, 4, 5, 6, 7, 8, 9, 10, 11, 12, 13, 14,15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or by more than 99% of the activity level of the wild type WPBF protein. In another embodiment, the WPBF protein with one or more mutations disclosed herein has no activity or zero activity as compared to wild type WPBF protein.

In yet another embodiment, the activity of the WPBF protein with one or more mutations disclosed herein is from 1-10% or from 10-30% or from 30-50% or from 50-70% or from 70-80% or from 80-90% or from 90-95% of the activity level of the wild type WPBF protein.

In one embodiment, the present application relates to a transgenic plant that comprises a transgene that encodes a polynucleotide, which down-regulates the expression of the WPBF gene and/or the activity of the WPBF protein. Examples of such polynucleotides include, but are not limited to, antisense polynucleotide, a sense polynucleotide, a catalytic polynucleotide, an artificial microRNA or a duplex RNA molecule. In one embodiment, the present application relates to a wheat plant comprising a transgene that reduces the expression of the WPBF gene and/or activity of the WPBF protein, where the wheat plant has increased fiber grains as compared to grains from a wild type plant.

In one embodiment, the present application relates to a plant with reduced expression of the WPBF gene and/or reduced activity of the WPBF protein, where reduced expression of the WPBF gene and/or reduced activity of the WPBF protein is achieved by genomic editing. In one embodiment, the present application relates to a wheat plant with a genome edited WPBF gene, where the wheat plant has increased fiber grains as compared to a wild type plant. In one embodiment, the present application relates to a wheat grain with a genome edited WPBF gene, where the wheat grain has increased fiber as compared to a wild type grains. In one embodiment, the method of introducing a human-induced mutation into the WPBF gene is carried out by genome editing.

Genome editing is a type of genetic engineering in which DNA is inserted, replaced, or removed from a genome using artificially engineered nucleases, or “molecular scissors.” The nucleases create specific double-stranded breaks (“DSBs”) at desired locations in the genome, and harness the cell's endogenous mechanisms to repair the induced break by natural processes of homologous recombination (“FIR”) and nonhomologous end-joining (“NHEJ”). There are currently four main families of engineered nucleases being used: Zinc finger nucleases (“ZFNs”), Transcription Activator-Like Effector Nucleases (“TALENs”), the CRISPR/Cas system, and engineered meganuclease with a re-engineered homing endonucleases.

The Clustered Regularly Interspaced Short Palindromic Repeats (“CRISPR”) Type II system is an RNA-Guided Endonuclease technology for genome engineering. There are two distinct components to this system: (1) a guide RNA and (2) a genome editing endonuclease, in this case the CRISPR associated (“Cas”) nuclease.

When the guide RNA and the genome editing endonuclease are expressed in the cell, the genomic target sequence can be modified or permanently disrupted. The guide RNA/genome editing endonuclease complex is recruited to the target sequence by the base-pairing between the guide RNA sequence and the complementary sequence of the target sequence in the genomic DNA. As used herein, the term “genome editing endonuclease”, “genome editing protein”, or “Cas endonuclease” refers to a protein such as a CRISPR (Clustered Regularly Interspaced Short Palindromic Repeats) associated nuclease. Non-limiting examples of CRISPR associated nucleases include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, homologs thereof, or modified versions, and endonuclease inactive versions thereof. CRISPR/Cas systems can be a type I, a type II, or a type III system. Use of such systems for gene editing has been widely described. For example, the use of CRISPR guide RNA in conjunction with CRISPR-Cas9 technology to target RNA is described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), which are hereby incorporated by reference in their entirety.

### Wheat Cultivars

In one embodiment, a wheat cultivar having at least one WPBF gene that is diploid, polyploid, tetraploid, or hexaploid may be used.

In another embodiment, the wheat is Triticum aestivum.

In one embodiment, any cultivar of wheat can be used to create mutations in an WPBF gene. In one embodiment, any cultivar of wheat can be used to create mutations in the WPBF gene of the A genome. In another embodiment, any cultivar of wheat can be used to create mutations in the WPBF gene of the B genome. In another embodiment, any cultivar of wheat can be used to create mutations in the WPBF gene of the D genome.

In one embodiment, any cultivar of wheat can be used as lines to cross WPBF mutations into different cultivars. In another embodiment, any cultivar of wheat having at least one WPBF gene may be used including but not limited to hard red spring wheat, hard white wheat, durum wheat, soft white spring, soft white winter wheat, hard red winter wheat, common wheat, club wheat, spelt wheat, emmer wheat, pasta wheat and turgidum wheat. Wheat varieties adapted to grow in different regions. For example, in the United States, different growing regions include the Pacific Northwest, the Desert Southwest, Northern Plains, Central Plains, and the Midwest and East.

In one embodiment, hard red spring wheat grown in the Northern Plains includes, but is not limited to, Barlow, Bullseye, Cabernet, Cal Rojo, Hank, Joaquin, Kelse, Lariat, Lassik, Malbec, Mika, PR 1404, Redwing, Summit 515, SY 314, Triple IV, Ultra, WB-Patron, WB-Rockland, Yecora Rojo, Accord, Aim, Anza, Baker, Beth Hashita, Bonus, Borah, Brim, Brooks, Buck Pronto, Butte 86, Cavalier, Challenger, Chief, Ciano T79, Colusa, Companion, Copper, Cuyama, Dash 12, Eldon, Elgin-ND, Enano, Express, Expresso, Jefferson, Genero F81, Grandin, Glenn, Helena 554, Hollis, Imuris T79, Inia 66R, Jerome, Kern, Len, Marshall, McKay, Nomad, Northwest 10, Oslo, Pavon F76, Pegasus, Pitic 62, Poco Red, Powell, Probrand 711, Probrand 751, Probrand 771, Probrand 775, Probred, Prointa Queguay, Prointa Quintal, Prosper, Rich, RSI 5, Sagittario, Scarlet, Serra, Shasta, Solano, Spillman, Sprite, Stander, Stellar, Steele, Stoa, Success, Summit, Sunstar 2, Sunstar King, Tadinia, Tammy, Tanori 71, Tara 2000, Tempo, Tesia T79, Topic, UI Winchester, Vance, Vandal, W444, Wampum, Wared, WB-Fuzion, Westbred 906R, Westbred 911, Westbred 926, Westbred 936, Westbred Discovery, Westbred Rambo, Yolo, Zeke, ND VitPro, Velva.

In another embodiment, hard white wheat includes, but is not limited to, Blanca Fuerte, Blanca Grande 515, Blanca Royale, Clear White, Patwin, Patwin 515, WB-Cristallo, WB-Paloma, WB-Perla, Alta Blanca, Blanca Grande, Delano, Golden Spike, ID377S, Klasic, Lochsa, Lolo, Macon, Otis, Phoenix, Pima 77, Plata, Pristine, Ramona 50, Siete Cerros 66, Vaiolet, and Winsome.

In yet another embodiment, durum wheat includes but is not limited to Crown, Desert King, Desert King HP, Duraking, Fortissimo, Havasu, Kronos, Maestrale, Normanno, Orita, Platinum, Q-Max, RSI 59, Saragolla, Tango, Tipai, Topper, Utopia, Volante, WB-Mead, Westmore, Aldente, Aldura, Altar 84, Aruba, Bittern, Bravadur, Candura, Cortez, Deluxe, Desert Titan, Durex, Durfort, Eddie, Germains 5003D, Imperial, Kofa, Levante, Matt, Mead, Mexicali 75, Minos, Modoc, Mohawk, Nudura, Ocotillo, Produra, Reva, Ria, Septre, Sky, Tacna, Titan, Trump, Ward, Westbred 803, Westbred 881, Westbred 883, Westbred 1000D, Westbred Laker, Westbred Turbo, and Yavaros 79.

In another embodiment, soft white spring wheat includes, but is not limited to, Alpowa, Alturas, Babe, Diva, JD, New Dirkwin, Nick, Twin,Whit, Blanca, Bliss, Calorwa, Centennial, Challis, Dirkwin, Eden, Edwall, Fielder, Fieldwin, Jubilee, Louise, Owens, Penawawa, Pomerelle, Sterling, Sunstar Promise, Super Dirkwin, Treasure, UI Cataldo, UI Pettit, Urquie, Vanna, Waduel, Waduel 94, Wakanz, Walladay, Wawawai, Whitebird, and Zak.

In still another embodiment, soft white winter wheat includes, but is not limited to, AP Badger, AP Legacy, Brundage 96, Bruneau, Cara, Goetze, Legion, Mary, Skiles, Stephens, SY Ovation, Tubbs, WB-Junction, WB-528, Xerpha, Yamhill, Barbee, Basin, Bitterroot, Bruehl, Castan, Chukar, Coda, Daws, Edwin, Eltan, Faro, Finch, Foote, Gene, Hill 81, Hiller, Hubbard, Hyak, Hyslop, Idaho 587, Kmor, Lambert, Lewjain, MacVicar, Madsen, Malcolm, Masami, McDermid, Moro, Nugaines, ORCF-101, ORCF-102, ORCF-103, Rod, Rohde, Rulo, Simon, Salute, Temple, Tres, Tubbs 06, UICF-Brundage, WB-523, and Weatherford.

In another embodiment, hard red winter wheat includes, but is not limited to, Andrews, Archer, Batum, Blizzard, Bonneville, Boundary, Declo, Debris, Finley, Garland, Hatton, Hoff, Longhorn, Manning, Meridian, Promontory, Vona, Wanser, Winridge.

In another embodiment, common wheat (hexaploid, free threshing), Triticum aestivum ssp aestivum includes, but is not limited to, Sonora, Wit Wolkoring, Chiddam Blanc De Mars, India-Jammu, Foisy.

In still another embodiment, spelt wheat (hexaploid, not free threshing), Triticum aestivum ssp spelta includes, but is not limited to, Spanish Spelt, Swiss Spelt.

In yet another embodiment, Emmer Wheat (tetraploid), Triticum turgidum ssp. dicoccum includes but is not limited to Ethiopian Blue Tinge.

In another embodiment, pasta wheat (tetraploid, free threshing), Triticum turgidum ssp durum includes but is not limited to Blue Beard, Durum-Iraq.

In yet another embodiment, Turgidum Wheat (tetraploid, free threshing), Triticum turgidum ssp turgidum includes but is not limited to Akmolinka, Maparcha.

In one embodiment, a cultivar of wheat having at least one WPBF gene with substantial percent identity to SEQ ID NO: 1, SEQ ID NO: 2, SEQ ID NO: 4, SEQ ID NO: 5, SEQ ID NO. 7, or SEQ ID NO. 8 may be used with the methods and compositions disclosed herein.

As used herein with regard to the wheat cultivars, “substantial percent identity” means that the DNA sequence of the gene is sufficiently similar to SEQ ID NO: 1, 2, 4, 5, 7, and 8 at the nucleotide level to code for a substantially similar protein, allowing for allelic differences (or alternate mRNA splicing) between cultivars. In accordance with one embodiment of the present application, “substantial percent identity” may be present when the percent identity in the coding region between the WPBF gene and SEQ ID NO: 1, 2, 4, 5, 7, and 8 is as low as about 85%, provided that the percent identity in the conserved regions of the gene is higher (e.g., at least about 90%). Preferably the percent identity in the coding region is 85-90%, more preferably 90-95%, and optimally, it is above 95%. Thus, one of skill in the art may prefer to utilize a wheat cultivar having commercial popularity or one having specific desired characteristics in which to create the WPBF-mutated wheat plants, without deviating from the scope and intent of the present application. Alternatively, one of skill in the art may prefer to utilize a wheat cultivar having few polymorphisms, such as an in-bred cultivar, in order to facilitate screening for mutations within one or more WPBF genes in accordance with the present application.

### Representative Methodology for Identification of WPBF Mutations

One of ordinary skill in the art will appreciate that numerous techniques and methods are available for generating mutations and/or human-induced mutations. One representative methodology is described below.

In order to create and identify the WPBF mutations and wheat plants disclosed herein, a method known as TILLING was utilized. See McCallum et al., “Targeted Screening for Induced Mutations,” Nature Biotechnology 18:455-457 (2000); McCallum et al., Plant Physiology, 123:439-442 (2000_; U.S. Patent Application Publication No. 2004/0053236; and U.S. Pat. No. 5,994,075, all of which are incorporated herein by reference in their entirety. In the basic TILLING methodology, plant materials, such as seeds, are subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with that mutation in the gene of interest.

In one embodiment, the tetraploid cultivar is Kronos. In other embodiments, the hexaploid cultivar is Express.

In one embodiment, seeds from wheat are mutagenized and then grown into M1 plants. The M1 plants are then allowed to self-pollinate and seeds from the M1 plant are grown into M2 plants, which are then screened for mutations in their WPBF loci. While M1 plants can be screened for mutations in accordance with alternative embodiments of the present application, one advantage of screening the M2 plants is that all somatic mutations correspond to germline mutations.

One of skill in the art will understand that a variety of wheat plant materials including, but not limited to, seeds, pollen, plant tissue or plant cells, may be mutagenized in order to create the WPBF-mutated wheat plants disclosed herein. However, the type of plant material mutagenized may affect when the plant DNA is screened for mutations. For example, when pollen is subjected to mutagenesis prior to pollination of a non-mutagenized plant, the seeds resulting from that pollination are grown into M1 plants. Every cell of the M1 plants will contain mutations created in the pollen, thus these M1 plants may then be screened for WPBF mutations instead of waiting until the M2 generation.

Mutagens that create primarily point mutations, short deletions (about 1 to about 200 nucleotides), insertions, transversions, and or transitions, such as chemical mutagens or radiation, such as ultraviolet light, x-rays and fast neutrons, may be used to create the mutations. Mutagens conforming with the method of the present application include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (DEB), and the like), 2-methoxy-6-chloro-9[3-(ethyl-2-chloro-ethyl)aminopropylamino] acridine dihydrochloride (ICR-170), sodium azide, formaldehyde, or combinations thereof.

In some embodiments, the method of increasing fiber in a wheat grain further comprises introducing a human-induced mutation into the WPBF gene using chemical mutagenesis. In some embodiments, the chemical is EMS.

Any suitable method of plant DNA preparation now known or hereafter devised may be used to prepare the wheat plant DNA for WPBF mutation screening. In one embodiment, prepared DNA from individual wheat plants are pooled in order to expedite screening for mutations in one or more WPBF genes of the entire population of plants originating from the mutagenized plant tissue. The size of the pooled group may be dependent upon the sensitivity of the screening method used. Preferably, groups of two or more individual wheat plants are pooled.

In another embodiment, after the DNA samples are pooled, the pools are subjected to WPBF sequence-specific amplification techniques, such as Polymerase Chain Reaction (PCR). Any primer specific to a WPBF locus or the sequences immediately adjacent to one of these loci may be utilized to amplify the WPBF sequences within the pooled DNA sample. In one embodiment, the primer is designed to amplify the regions of the WPBF locus where useful mutations are most likely to arise. The primer may be designed to detect exonic regions of one or more WPBF genes. Additionally, the primer may be known to target known polymorphic sites to design genome specific primers in order to ease screening for point mutations in a particular genome. To facilitate detection of PCR products on a gel, the PCR primer may be labeled using any conventional or hereafter devised labeling method.

In one embodiment, primers are designed based upon the WPBF genes (SEQ ID NOs: 1, 2, 4, 5, 7, and 8).

In another embodiment, the PCR amplification products may be screened for WPBF mutations using any method that identifies nucleotide differences between wild type and mutant sequences. These may include, for example and without limitation, sequencing, denaturing high pressure liquid chromatography (dHPLC), constant denaturant capillary electrophoresis (CDCE), temperature gradient capillary electrophoresis (TGCE), or by fragmentation using enzymatic cleavage, such as used in the high throughput method described by Colbert et al., “High-Throughput Screening for Induced Mutations,” Plant Physiology 126:480-484 (2001), which is hereby incorporated by reference in its entirety. Preferably, the PCR amplification products are incubated with an endonuclease that preferentially cleaves mismatches in heteroduplexes between wild type and mutant sequences.

In another embodiment, human-induced mutations may be identified by next generation sequencing such as described in Krasileva et al., “Uncovering Hidden Variation in Polyploid Wheat,” Proc. Nat. Acad. Sci. 114-E913-E921 (2017), which is hereby incorporated by reference in its entirety.

In another embodiment, cleavage products are electrophoresed using an automated sequencing gel apparatus, and gel images are analyzed with the aid of a standard commercial image-processing program.

In yet another embodiment, once an M2 plant having a mutated WPBF gene sequence is identified, the mutations are analyzed to determine their effect on the expression, translation, and/or activity of a WPBF enzyme. In one embodiment, the PCR fragment containing the mutation is sequenced, using standard sequencing techniques, to determine the exact location of the mutation in relation to the overall WPBF sequence. Each mutation is evaluated to predict its impact on protein function (i.e., from completely tolerated to causing loss-of-function) using bioinformatics tools such as SIFT (Ng and Henikoff, “SIFT: Predicting Amino Acid Changes that Affect Protein Function,” Nucleic Acids Research 31:3812-3814 (2003), PS SM (Henikoff and Henikoff, “Using Substitution Probabilities to Improve Position-Specific Scoring Matrices,” Computer Applications in the Biosciences 12:135-143 (1996)), and PARSESNP (Taylor and Greene, “PARSESNP: A Tool for the Analysis of Nucleotide Polymorphisms,” Nucleic Acids Research 31:3808-3811 (2003), which are hereby incorporated by reference in their entirety). For example, a SIFT score that is less than 0.05 or a large change in PSSM score (e.g., 10 or above) indicate a mutation that is likely to have a deleterious effect on protein function. These mutations are indicated as severe missense mutations in Tables 1-3. These programs are known to be predictive, and it is understood by those skilled in the art that the predicted outcomes are not always accurate.

In another embodiment, if the initial assessment of a mutation in the M2 plant indicates it to be of a useful nature and in a useful position within a WPBF gene, then further phenotypic analysis of the wheat plant containing that mutation may be pursued. In hexaploid wheat, mutations in each of the A, B, and D genomes may need to be combined before a phenotype can be detected. In tetraploid wheat, mutations in both the A and B genome mutations may need to be combined before a phenotype can be detected. In one embodiment, the mutation-containing plant can be backcrossed or outcrossed two times or more to eliminate background mutations at any generation. Then, the backcrossed or outcrossed plant can be self-pollinated or crossed to create plants that are homozygous for the WPBF mutations.

Several physical characteristics of these homozygous WPBF mutant plants are assessed to determine if the mutation(s) results in a useful phenotypic change in the wheat plant without resulting in undesirable negative effects, such as significantly reduced seed yields.

### Methods of Producing a Wheat Plant

In another aspect, the present application relates to a method for producing a wheat plant with increased fiber grains. In one embodiment, the present application relates to a method for producing a wheat plant with increased resistant starch. In another embodiment, the present application relates to a method for producing a wheat plant with increased fructans. In another embodiment, the present application relates to a method for producing plants with increased insoluble fiber. In another embodiment, the present application relates to a method for producing plants with increased soluble fiber.

In another embodiment, the present application relates to a method of out-crossing WPBF gene mutations to wild type plants.

In still another embodiment, the present application relates to a method for producing a plant having reduced activity of one or more WPBF proteins as compared to the wild type wheat plants.

In one embodiment, the method comprises inducing at least one human-induced mutation in at least one copy of a WPBF gene in plant material or plant parts from a parent plant; growing or using the mutagenized plant material to produce progeny plants; analyzing mutagenized plant material and/or progeny plants to detect at least one mutation in at least one copy of a WPBF gene; and selecting progeny plants that have at least one mutation in at least one copy of a WPBF gene.

In another embodiment, the method further comprises crossing progeny plants that have at least one mutation in at least one copy of a WPBF gene with other progeny plants that have at least one mutation in a different copy of a WPBF gene. The process of identifying progeny plants with mutations and crossing said progeny plants with other progeny plants, which have mutations, can be repeated to produce progeny wheat plants with reduced WPBF/WPBF activity.

In one embodiment, the level of activity of the WPBF protein in the wheat plant is reduced to 0-2%, 2-5%, 5-7%, 7-10%, 10-15%, 15-20%, 20-25%, 25-30%, 30-35%, 35-40%, 40-45%, 45-50%, 50-60%, 60-70%, 70-80%, 80-90%, 90-95%, or 95-99% of the level of activity of the WPBF protein in the wild type plant.

Methods of Producing a Plant with One or More Human-Induced Mutations in the WPBF Gene in More than One Genome

The present application also relates to other methods of producing plants, including the following: inducing at least one human-induced mutation in at least one copy of a WPBF gene in plant material from a parent plant that comprises a mutation in a WPBF gene; growing or using the mutagenized plant material to produce progeny plants; and selecting progeny wheat plants that have at least one mutation in at least two copies of a WPBF gene. For example, the parent plant may have a mutation in a WPBF gene of the A genome. The selected progeny plants may have a mutation in a WPBF gene of the A genome and one or more mutations in the WPBF gene of the B genome. The selected progeny plants may have a mutation in a WPBF gene of the A genome and one or more mutations in the WPBF gene of the B genome. The selected progeny plants may have a mutation in a WPBF gene of the A genome and one or more mutations in the WPBF gene of the D genome. These examples are provided merely for clarification and should not limit the methods disclosed herein.

In yet another embodiment, the present application relates to a method for producing a plant comprising inducing at least one human-induced mutation in at least one copy of a WPBF gene in plant material from a parent plant that comprises at least one mutation in two WPBF genes; growing or using the mutagenized plant material to produce progeny plants; and selecting progeny plants that have at least one mutation in three copies of a WPBF gene. In this embodiment, there would be at least one mutation in the WPBF gene of the A, B, and D genomes.

In another embodiment, the present application relates to a method for producing a wheat plant comprising crossing a first plant that has at least one human-induced mutation in a first WPBF gene with a second plant that has at least one human-induced mutation in a second WPBF gene; and selecting progeny plants that have at least one mutation in at least two copies of a WPBF gene.

In another embodiment, the present application relates to a method for producing a plant comprising crossing a first plant that has at least one human-induced mutation in a first and second WPBF gene with a second plant that has at least one human-induced mutation in a third WPBF gene; and selecting progeny plants that have at least one mutation in all three copies of a WPBF gene. In this embodiment, there would be at least one mutation in the WPBF gene of the A, B, and D genomes.

### Wheat Plant, Wheat Seed, and Parts of Wheat Plant

In one embodiment, a wheat plant with increased fiber grains is produced according to the methods disclosed herein. In one embodiment, a wheat plant with reduced low molecular weight glutenins is produced according to the methods disclosed herein. In one embodiment, a wheat plant with reduced gliadins is produced according to the methods disclosed herein. In yet another embodiment, a wheat plant with increased or unaltered high molecular weight glutenins is produced according to the methods disclosed herein.

In another embodiment, the wheat plant, wheat seed, or parts of a wheat plant have one or more mutations in a WPBF gene or a modified WPBF gene. In another embodiment, the wheat plant, wheat seed, or parts of a wheat plant have one or more mutations in WPBF genes.

In another embodiment, the present application relates to a wheat plant, wheat seed, or parts of a wheat plant comprising one or more human-induced mutations in the WPBF gene. In another embodiment, the present application relates to a wheat plant, wheat seed, or parts of a wheat plant comprising at least one human-induced mutation in the WPBF gene in each of two genomes. In still another embodiment, the present application relates to a wheat plant, wheat seed, or parts of a wheat plant comprising at least one human-induced mutation in the WPBF gene in each of three genomes.

In one embodiment, the wheat plant, wheat seed, or parts of a wheat plant comprises one or more human-induced mutations in both alleles of the WPBF gene in the A genome. In another embodiment, the human-induced mutations are identical in both alleles of the WPBF gene of the A genome.

In one embodiment, the wheat plant, wheat seed, or parts of a wheat plant comprises one or more human-induced mutations in both alleles of the WPBF gene in the B genome. In another embodiment, the human-induced mutations are identical in both alleles of the WPBF gene of the B genome.

In one embodiment, the wheat plant, wheat seed, or parts of a wheat plant comprises one or more human-induced mutations in both alleles of the WPBF gene in the D genome. In another embodiment, the human-induced mutations are identical in both alleles of the WPBF gene of the D genome.

In another embodiment, the wheat plant, wheat seed or parts of the wheat plant comprise a polynucleotide with one or more human-induced mutations listed in Table 1 and has 85%, 86%, 87%, 88%, 89%, 90%, 91%, 92%, 93%, 94%, 95%, 96%, 97%, 98%, 99%, or greater than 99% identity or similarity to SEQ ID NO: 2.

In still another embodiment, the wheat plant, wheat seed or parts of a wheat plant comprise a polynucleotide with one or more human-induced mutations listed in Table 1 that codes for a WPBF protein, where the WPBF protein comprises one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 3.

In another embodiment, the wheat plant, wheat seed or parts of the wheat plant comprise a polynucleotide with one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 5.

In still another embodiment, the wheat plant, wheat seed or parts of a wheat plant comprise a polynucleotide with one or more human-induced mutations that codes for a WPBF protein, where the WPBF protein comprises one or more human-induced mutations and has 85%, 86%, 87%, 88%, 89%, 90%, 91%, 92%, 93%, 94%, 95%, 96%, 97%, 98%, 99% or greater than 99% identity or similarity to SEQ ID NO: 6.

In another embodiment, the wheat plant, wheat seed or parts of the wheat plant comprise a polynucleotide with one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 8.

In still another embodiment, the wheat plant, wheat seed, or parts of a wheat plant comprise a polynucleotide with one or more human-induced mutations that codes for a WPBF protein, where the WPBF protein comprises one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 9.

In another embodiment, the wheat plant, wheat seed or parts of a wheat plant has one or more mutations in the WPBF gene including but not limited to one or more mutations enumerated in Tables 1-3 and corresponding mutations in the homoeologues. A wheat plant, wheat seed or parts of a wheat plant can be generated having 1, 2, 3, 4, 5, 6, 7, 8, 9, 10, 11, 12, 13, 14, 15, 16, 17, 18, 19, 20, 21, 22, 23, 24, 25, or greater than 25 of the mutations disclosed herein including but not limited to the mutations disclosed in Tables 1-3, as well as mutations in corresponding homoeologues.

### Grain, Flour, and Starch

In another embodiment, the present application relates to a wheat grain, flour, or starch comprising one or more human-induced human-induced mutations in the WPBF gene or a modified WPBF gene. In another embodiment, the present application relates to wheat grain comprising an embryo, where the embryo comprises one or more human-induced mutations in a WPBF gene or a modified WPBF gene.

In another embodiment, the wheat grain, flour or starch comprises one or more human-induced mutations in the WPBF genes including, but not limited to, the mutations recited in Tables 1-3 and the corresponding mutations in homologues and homoeologues.

In still another embodiment, the present application relates to a wheat grain or flour comprising at least one human-induced mutation in the WPBF gene in one, two, or three genomes.

In still another embodiment, the present application relates to a wheat grain, flour, or starch comprising one or more human-induced mutations in the WPBF gene of the A genome. In another embodiment, the human-induced mutations are identical in both alleles of the WPBF gene of the A genome.

In one embodiment, the wheat grain, flour, or starch comprises one or more human-induced mutations in both alleles of the WPBF gene in the B genome. In another embodiment, the human-induced mutations are identical in both alleles of the WPBF gene of the B genome.

In one embodiment, the wheat grain, flour, or starch comprises one or more human-induced mutations in both alleles of the WPBF gene in the D genome. In another embodiment, the human-induced mutations are identical in both alleles of the WPBF gene of the D genome.

In one embodiment, the present application relates to wheat grain, wheat flour or starch comprising a polynucleotide of the WPBF gene in the A genome with one or more human-induced mutations listed in Table 1 and corresponding to SEQ ID NO: 2. In another embodiment, the wheat grain or wheat flour comprise a polynucleotide with one or more human-induced mutations listed in Table 1 and has 85%, 86%, 87%, 88%, 89%, 90%, 91%, 92%, 93%, 94%, 95%, 96%, 97%, 98%, 99%, or greater than 99% identity or similarity to SEQ ID NO: 2.

In still another embodiment, wheat grain, wheat flour, or starch comprise a polynucleotide with one or more human-induced mutations listed in Table 1 that codes for a WPBF protein, wherein the WPBF protein comprises one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 3.

In one embodiment, the present application relates to wheat grain, wheat flour, or starch comprising a polynucleotide of the WPBF gene in the B genome with one or more human-induced mutations listed in Table 2 and corresponding to SEQ ID NO: 5. In another embodiment, the wheat grain or wheat flour comprise a polynucleotide with one or more human-induced mutations listed in Table 2 and has 85%, 86%, 87%, 88%, 89%, 90%, 91%, 92%, 93%, 94%, 95%, 96%, 97%, 98%, 99%, or greater than 99% identity or similarity to SEQ ID NO: 5.

In still another embodiment, wheat grain, wheat flour, or starch comprise a polynucleotide with one or more human-induced mutations listed in Table 2 that codes for a WPBF protein, where the WPBF protein comprises one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 6.

In one embodiment, the present application relates to wheat grain, wheat flour, or starch comprising a polynucleotide of the WPBF gene in the D genome with one or more human-induced mutations listed in Table 3 and corresponding to SEQ ID NO: 8. In another embodiment, the wheat grain or wheat flour comprise a polynucleotide with one or more human-induced mutations listed in Table 3 and has 85%, 86%, 87%, 88%, 89%, 90%, 91%, 92%, 93%, 94%, 95%, 96%, 97%, 98%, 99%, or greater than 99% identity or similarity to SEQ ID NO: 8.

In still another embodiment, wheat grain, wheat flour, or starch comprise a polynucleotide with one or more human-induced mutations listed in Table 3 that codes for a WPBF protein, where the WPBF protein comprises one or more human-induced mutations and has 85%, 86%, 87%, 88%, 89%, 90%, 91%, 92%, 93%, 94%, 95%, 96%, 97%, 98%, 99%, or greater than 99% identity or similarity to SEQ ID NO: 9.

In still another embodiment, the present application relates to wheat grain or flour comprising an endosperm and a reduced gene expression level, activity, or expression level and activity of the WPBF gene as compared to wild type wheat grain or flour.

In yet another embodiment, the present application relates to wheat grain or flour with one or more mutations in the WPBF gene or a modified WPBF gene exhibiting increased fiber as compared to wild type wheat grain or flour. In another embodiment, wheat grain or flour with one or more mutations in the WPBF gene or a modified WPBF gene exhibits from 1-5%, 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, or greater than 95% increased fiber as compared to wild type grain or flour.

In one embodiment, grains disclosed herein may contain embryos that are larger than wild type grains. In one embodiment, the grains disclosed herein may contain embryos that are from 1-5%, or from 5-10%, or 10-15%, or from 15-20%, or from 20-25%, or from 25-50%, or from 50-75%, or from 75-95% larger than embryos of wild type grains.

In one embodiment, the grains disclosed herein may contain embryos that are at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% greater in size than embryos of wild type gains.

### Food Products

In one embodiment, the present application is directed to a flour or other product produced from the grain or flour discussed above. In another embodiment, the flour, the coarse fraction, or purified starch may be a component of a food product.

The food product includes, but is not limited to, a bagel, a biscuit, a bread, a bun, a croissant, a dumpling, an English muffin, a muffin, a pita bread, a quickbread, a flat bread, a sourdough bread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food, and pet food.

In some embodiments, the flour is a dry mix product such as, but not limited to a mix for a muffin, a waffle, a cookie, a pancake, a biscuit, a cake, a pizza crust, a bagel, a bread, and a pie crust.

In one embodiment, the flour is a whole grain flour (e.g., an ultrafine-milled whole grain flour, such as an ultrafine-milled whole grain wheat flour). In one embodiment, the whole grain flour includes a refined flour constituent (e.g., refined wheat flour or refined flour) and a coarse fraction (e.g., an ultrafine-milled coarse fraction). Refined wheat flour may be flour which is prepared, for example, by grinding and bolting (sifting) cleaned wheat. The Food and Drug Administration (FDA) requires flour to meet certain particle size standards in order to be included in the category of refined wheat flour. The particle size of refined wheat flour is described as flour in which not less than 98% passes through a cloth having openings not larger than those of woven wire cloth designated “212 micrometers (U.S. Wire 70).”

In another embodiment, the coarse fraction includes at least one of bran and germ. For instance, the germ is an embryonic plant found within the wheat kernel. The germ includes lipids, fiber, vitamins, protein, minerals and phytonutrients, such as flavonoids. The bran may include several cell layers and has a significant amount of lipids, fiber, vitamins, protein, minerals and phytonutrients, such as flavonoids.

For example, the coarse fraction or whole grain flour or refined flour of the present application may be used in various amounts to replace refined or whole grain flour in baked goods, snack products, and food products. The whole grain flour (i.e. ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In another embodiment, the whole grain flour or coarse fraction or refined flour may be a component of a nutritional supplement. The nutritional supplement may be a product that is added to the diet containing one or more ingredients, typically including: vitamins, minerals, herbs, amino acids, enzymes, antioxidants, herbs, spices, probiotics, extracts, prebiotics and fiber.

In a further embodiment, the nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. Because of the high nutritional content of the endosperm of the present application, there may be many uses that confer numerous benefits to an individual, including, delivery of fiber and other essential nutrients, increased digestive function and health, weight management, blood sugar management, heart health, diabetes risk reduction, potential arthritis risk reduction, and overall health and wellness for an individual.

In still another embodiments, the whole grain flour or coarse fraction or refined flour may be a component of a dietary supplement. The Code of Federal Regulations defines a dietary supplement as a product that is intended to supplement the diet and contains one or more dietary ingredients including: vitamins, minerals, herbs, botanicals, amino acids, and other substances or their constituents; is intended to be taken by mouth as a pill, capsule, tablet, or liquid; and is labeled on the front panel as being a dietary supplement.

In yet another embodiment, the whole grain flour or coarse fraction or refined flour may be a fiber supplement or a component thereof. The fiber supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage.

In another embodiment, the whole grain flour or coarse fraction or refined flour may be included as a component of a digestive supplement. The whole grain flour or coarse fraction or refined flour may be a component of a digestive supplement alone or in combination with one or more prebiotic compounds and/or probiotic organisms. Prebiotic compounds are non-digestible food ingredients that may beneficially affect the host by selectively stimulating the growth and/or the activity of a limited number of microorganisms in the colon. Examples of prebiotic compounds within the scope of the present application, may include, but are not limited to, oligosaccharides and inulins.

Probiotics are microorganisms which, when administered in adequate amounts, confer a health benefit on the host. Probiotic organisms include, but are not limited to, Lactobacillus, Bifidobacteria, Escherichia, Clostridium, Lactococcus, Streptococcus, Enterococcus, and Saccharomyces.

In yet another embodiment, the whole grain flour or coarse fraction or refined flour may be included as a component of a functional food. The Institute of Food Technologists defines functional foods as, foods and food components that provide a health benefit beyond basic nutrition. This includes conventional foods, fortified, enriched, or enhanced foods, and dietary supplements. The whole grain flour and coarse fraction or refined flour include numerous vitamins and minerals, have high oxygen radical absorption capacities, and are high in fiber, making them ideally suited for use in/as a functional food.

In another embodiment, the whole grain flour or coarse fraction or refined flour may be used in medical foods. Medical food is defined as a food that is formulated to be consumed or administered entirely under the supervision of a physician and which is intended for the specific dietary management of a disease or condition for which distinctive nutritional requirements, based on recognized scientific principles, are established by medical evaluation. The nutrient contents and antioxidant capacities of the whole grain flour and coarse fraction or refined flour make them ideal for use in medical foods.

In yet another embodiment, the whole grain flour or coarse fraction or refined flour may also be used in pharmaceuticals. The whole grain flour and coarse fraction or refined flour are high in fiber and have a very fine granulation making them suitable for use as a carrier in pharmaceuticals.

In still another embodiment, delivery of the whole grain flour or coarse fraction or refined flour as a nutritional supplement, dietary supplement or digestive supplement is contemplated via delivery mechanisms where the whole grain flour or coarse fraction is the single ingredient or one of many nutritional ingredients. Examples of delivery mechanisms include but are not limited to: instant beverage mixes, ready-to-drink beverages, nutritional bars, wafers, cookies, crackers, gel shots, capsules, and chews.

In yet another embodiment, a milling process may be used to make a multi-wheat flour, or a multi-grain coarse fraction. In one embodiment, bran and germ from one type of wheat may be ground and blended with ground endosperm or whole grain wheat flour of another type of wheat. Alternatively bran and germ of one type of grain may be ground and blended with ground endosperm or whole grain flour of another type of grain.

In still another embodiment, bran and germ from a first type of wheat or grain may be blended with bran and germ from a second type of wheat or grain to produce a multi-grain coarse fraction. It is contemplated that the present application encompasses mixing any combination of one or more of bran, germ, endosperm, and whole grain flour of one or more grains. This multi-grain, multi-wheat approach may be used to make custom flour and capitalize on the qualities and nutritional contents of multiple types of grains or wheats to make one flour.

The whole grain flour of the present application may be produced via a variety of milling processes. One exemplary process involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like.

After grinding, the grain is discharged and conveyed to a sifter. Any sifter known in the art for sifting a ground particle may be used. Material passing through the screen of the sifter is the whole grain flour of the present application and requires no further processing. Material that remains on the screen is referred to as a second fraction. The second fraction requires additional particle reduction. Thus, this second fraction may be conveyed to a second passage grinder.

After grinding, the second fraction may be conveyed to a second sifter. Material passing through the screen of the second sifter is the whole grain flour. The material that remains on the screen is referred to as the fourth fraction and requires further processing to reduce the particle size. The fourth fraction on the screen of the second sifter is conveyed back into either the first passage grinder or the second passage grinder for further processing via a feedback loop.

It is contemplated that the whole grain flour, coarse fraction, purified starch, and/or grain products of the present application may be produced by a number of milling processes known in the art.

### Plant Breeding

In another embodiment, the present application is directed to methods for plant breeding using wheat plants and plant parts with one or more human-induced mutations in the WPBF gene.

One such embodiment is the method of crossing a wheat variety with one or more human-induced mutations in the WPBF gene with another variety of wheat to form a first generation population of F1 plants. The population of first generation F1 plants produced by this method is also an embodiment of the present application. This first generation population of F1 plants will comprise an essentially complete set of the alleles of a wheat variety with one or more human-induced mutations in the WPBF gene. One of ordinary skill in the art can utilize either breeder books or molecular methods to identify a particular F1 plant produced using a wheat variety with one or more human-induced mutations in the WPBF gene, and any such individual plant is also encompassed by this present application. These embodiments also cover use of transgenic or backcross conversions of wheat varieties with one or more mutations in the WPBF gene to produce first generation F1 plants.

In another embodiment, the present application relates to a method of developing a progeny wheat plant. A method of developing a progeny wheat plant comprises crossing a wheat variety with one or more human-induced mutations in the WPBF gene with a second wheat plant and performing a breeding method. A specific method for producing a line derived from a wheat variety with one or more human-induced mutations in the WPBF gene is as follows.

One of ordinary skill in the art would cross a wheat variety with one or more human-induced mutations in the WPBF gene with another variety of wheat, such as an elite variety. The F1 seed derived from this cross would be grown to form a homogeneous population. The F1 seed would contain one set of the alleles from a wheat variety with one or more human-induced mutations in the WPBF gene and one set of the alleles from the other wheat variety.

The F1 genome would be made-up of 50% of a wheat variety with one or more human-induced mutations in the WPBF gene and 50% of the other elite variety. The F1 seed would be grown to form F2 seed. The F1 seed could be allowed to self, or bred with another wheat cultivar.

On average the F2 seed would have derived 50% of its alleles from a wheat variety with one or more human-induced mutations in the WPBF gene and 50% from the other wheat variety, but various individual plants from the population would have a much greater percentage of their alleles derived from a wheat variety with one or more human-induced mutations in the WPBF gene (Wang and Bernardo, “Variance of Marker Estimates of Parental Contribution to F2 and BC1-Derived Inbreds,” Crop Sci. 40:659-665 (2000) and Bernardo and Kahler, “North American Study on Essential Derivation in Maize: Inbreds Developed without and with Selection from F2 Populations,” Theor. Appl. Genet. 102:986-992 (2001), which are hereby incorporated by reference in their entirety).

The F2 seed would be grown and selection of plants would be made based on visual observation and/or measurement of traits and/or marker assisted selection. The wheat variety with one or more human-induced mutations in the WPBF gene-derived progeny that exhibit gene-derived traits would be selected and each plant would be harvested separately. This F3 seed from each plant would be grown in individual rows and allowed to self. Then selected rows or plants from the rows would be harvested and threshed individually. The selections would again be based on visual observation and/or measurements for desirable traits of the plants, such as one or more of the desirable wheat variety with one or more mutations in the WPBF gene-derived traits.

The process of growing and selection would be repeated any number of times until a homozygous wheat variety with one or more human-induced mutations in the WPBF gene-derived wheat plant is obtained. The homozygous wheat variety with one or more human-induced mutations in the WPBF gene-derived wheat plant would contain desirable traits derived from the wheat variety with one or more human-induced mutations in the WPBF gene, some of which may not have been expressed by the other original wheat variety to which the wheat variety with one or more human-induced mutations in the WPBF gene was crossed and some of which may have been expressed by both wheat varieties but now would be at a level equal to or greater than the level expressed in the wheat variety with one or more human-induced mutations in the WPBF gene.

The breeding process, of crossing, selfing, and selection may be repeated to produce another population of wheat variety with one or more human-induced mutations in the WPBF gene-derived wheat plants with, on average, 25% of their genes derived from wheat variety with one or more human-induced mutations in the WPBF gene, but various individual plants from the population would have a much greater percentage of their alleles derived from the wheat variety with one or more human-induced mutations in the WPBF gene. Another embodiment of the present application is a homozygous wheat variety with one or more human-induced mutations in the WPBF gene-derived wheat plant that has been crossed with another wheat plant with one or more human-induced mutations in the WPBF gene-derived traits.

### Mutations as Markers

Genetic markers are the biological features that are determined by allelic forms of genes or genetic loci and can be transmitted from one generation to another, and thus they can be used as experimental probes or tags to keep track of an individual, a plant, a tissue, a cell, a nucleus, a chromo-some or a gene.

SNPs provide the ultimate/simplest form of molecular markers as a single nucleotide base is the smallest unit of inheritance, and thus they can provide the maximum number of markers. SNPs occur very commonly in animals and plants. Typically, SNP frequencies are in a range of one SNP every 100-300 base pairs in plants. SNPs may be present within coding sequences of genes, non-coding regions of genes or in the intergenic regions between genes at different frequencies in different chromosome regions.

SNPs are co-dominant markers, often linked to genes and present in the simplest/ultimate form for polymorphism, and thus they have become very attractive and potential genetic markers in genetic study and breeding. Moreover, SNPs can be very easily automated and quickly detected, with a high efficiency for detection of polymorphisms.

In one embodiment, the mutation in WPBF is a single nucleotide polymorphism. In one embodiment, the present application relates to mutations in the WPBF gene which, according to one embodiment, are single nucleotide polymorphisms that can be used as markers in plant breeding. The mutations in the WPBF gene are causative and their segregation can be followed using, for example, KASP probes.

PCR-based KASP™ genotyping assay is a homogeneous, fluorescence (FRET) based assay that enables accurate bi-allelic discrimination of known SNPs and InDels. A key feature of PCR-based KASP technology is the use of a universal FRET cassette reporter system that eliminates the need for costly dual-labelled probes. The allele-specific forward primers each have a proprietary tail sequence that corresponds with one of two FRET cassettes: one label with FAM dye and the other with HEX dye. Bi-allelic discrimination is achieved through the competitive binding of the two allele-specific forward primers.

Another aspect of the present application relates to a method of producing dietary fiber. This method involves providing a wheat plant comprising a human-induced mutation in a Wheat Prolamin-box Binding Factor (WPBF) gene and introducing a human-induced mutation into the WPBF gene, where the mutation causes the wheat plant to produce grain with increased fiber compared to a wheat plant without the mutation and obtaining fiber from the grain of the wheat plant.

This aspect of the present application can be carried out with any of the embodiments described herein.

A further aspect of the present application relates to a wheat plant capable of producing grain with elevated fiber content, the wheat plant comprising a human-induced mutation in a Wheat Prolamin-box Binding Factor (WPBF) gene, where the mutation is effective to elevate fiber in the grain of a wheat plant compared to grain from a wheat plant without the mutation.

This aspect of the present application can be carried out with any of the embodiments described herein.

Another aspect of the present application relates to a method of selecting wheat grain with high fiber content. This method involves introducing a human-induced mutation into a wheat plant or plant part, producing a wheat plant having the mutation, where the wheat plant producing grain. The method further involves identifying grain from the produced wheat plant having an embryo larger than an embryo of wheat grain from a wild-type wheat plant, and selecting the grain with the larger embryo as wheat grain with higher fiber content.

This aspect of the present application can be carried out with any of the embodiments described herein.

### EXAMPLES

### Example 1—Mutagenesis of Wheat Seeds

In accordance with one exemplary embodiment of the present application, wheat seeds of the hexaploid cultivar (Triticum aestivum) Express or tetraploid cultivar Kronos were vacuum infiltrated in H₂O 2O (approximately 1,000 seeds/100 ml H₂O 2O for approximately 4 minutes). The seeds were then placed on a shaker (45 rpm) in a fume hood at room temperature. The mutagen ethyl methanesulfonate (EMS) was added to the imbibing seeds to final concentrations ranging from about 0.75% to about 1.2% (v/v). Following an 18-hour incubation period, the EMS solution was replaced 4 times with fresh H₂O. The seeds were then rinsed under running water for about 4-8 hours. Finally, the mutagenized seeds were planted (96/tray) in potting soil and allowed to germinate indoors. Plants that were four to six weeks old were transferred to the field to grow to fully mature M1 plants. The mature M1 plants were allowed to self-pollinate and then seeds from the M1 plant were collected and planted to produce M2 plants.

DNA from the M2 plants produced in accordance with the above description was extracted and prepared in order to identify which M2 plants carried a mutation at one or more of their WPBF loci. The M2 plant DNA was prepared using the methods and reagents contained in the Qiagen® (Valencia, CA) DNeasy® 96 Plant Kit. Approximately 50 mg of frozen plant sample was placed in a sample tube with a tungsten bead, frozen in liquid nitrogen and ground 2 times for 1 minute each at 20 Hz using the Retsch® Mixer Mill MM 300. Next, 400 μl of solution AP1 [Buffer AP1, solution DX and RNAse (100 mg/ml)] at 80° C. was added to the sample. The tube was sealed and shaken for 15 seconds. Following the addition of 130 μl Buffer AP2, the tube was shaken for 15 seconds. The samples were placed in a freezer at minus 20° C. for at least 1 hour. The samples were then centrifuged for 20 minutes at 5,600×g. A 400 μl aliquot of supernatant was transferred to another sample tube. Following the addition of 600 μl of Buffer AP3/E, this sample tube was capped and shaken for 15 seconds. A filter plate was placed on a square well block and 1 ml of the sample solution was applied to each well and the plate was sealed. The plate and block were centrifuged for 4 minutes at 5,600×g. Next, 800 μl of Buffer AW was added to each well of the filter plate, sealed and spun for 15 minutes at 5,600×g in the square well block. The filter plate was then placed on a new set of sample tubes and 80 μl of Buffer AE was applied to the filter. It was capped and incubated at room temperature for 1 minute and then spun for 2 minutes at 5600×g. This step was repeated with an additional 80 μl Buffer AE. The filter plate was removed and the tubes containing the pooled filtrates were capped. The individual samples were then normalized to a DNA concentration of 5 to 10 ng/μ1.

### Tilling

The M2 wheat DNA was pooled into groups of two individual plants. The DNA concentration for each individual within the pool was approximately 2 ng/ul with a final concentration of 4 ng/μ1 for the entire pool. Then, 5 μl of the pooled DNA samples (or 20 ng wheat DNA) was arrayed on microtiter plates and subjected to gene-specific PCR.

PCR amplification was performed in 15 IA volumes containing 20 ng pooled DNA, 0.75×ExTaq buffer (Clonetech, Mountain View, CA), 1.1 mM additional MgCl₂, 0.3 mM dNTPs, 0.3 μM primers, 0.009 U Ex-Taq DNA polymerase (Clonetech, Mountain View, CA), 0.02 units DyNAzyme II DNA Polymerase (Thermo Scientific), and if necessary 0.33M Polymer-Aide PCR Enhancer (Sigma-Aldrich®). PCR amplification was performed using an MJ Research® thermal cycler as follows: 95° C. for 2 minutes; 8 cycles of “touchdown PCR” (94° C. for 20 second, followed by annealing step starting at 70-68° C. for 30 seconds and decreasing 1° C. per cycle, then a temperature ramp of 0.5° C. per second to 72° C. followed by 72° C. for 1 minute); 25-45 cycles of 94° C. for 20 seconds, 63 or 65° C. for 30 seconds, ramp 0.5° C./sec to 72° C., 72° C. for 1-2 minutes; 72° C. for 8 minutes; 98° C. for 8 minutes; 80° C. for 20 seconds; 60 cycles of 80° C. for 7 seconds −0.3 degrees/cycle.

PCR products (2-4 IA) were digested in 96-well plates. 3 μl of a solution containing 6 mM HEPES [4-(2-hydroxyethyl)-1-piperazineethanesulfonic acid] (pH 7.0), 6 mM MgCl₂, 6 mM NaCl, 0.012×Triton® X-100, 0.03 mg/ml of bovine serum albumin, 0.5×T-Digest Buffer [Advanced Analytical Technologies, Inc (AATI), Ames, IA], 0.912 U each of Surveyor® Endonuclease and Enhancer (Transgenomic®, Inc.), and 0.5×dsDNA Cleavage Enzyme (AATI, Ames, IA) was added to the PCR product. Digestion reactions were incubated at 45° C. for 45 minutes. The specific activity of the Surveyor enzyme was 800 units/μ1, where a unit was defined by the manufacturer as the amount of enzyme required to produce 1 ng of acid-soluble material from sheared, heat denatured calf thymus DNA at pH 8.5 in one minute at 37° C. Reactions were stopped by addition of 20 IA of Dilution Buffer E (AATI, Ames, IA) or 1×TE. The reactions were stored in the freezer until they were run on the Fragment Analyzer™ (AATI, Ames, IA) Capillary Electrophoresis System. Samples were run on the Fragment Analyzer™ utilizing the DNF-920-K1000T Mutation Discovery Kit (AATI, Ames, IA) according to the manufacturer's protocol.

After electrophoresis, the assays were analyzed using PROSize® 2.0 Software (AATI, Ames, IA). The gel image showed sequence-specific pattern of background bands common to all 96 lanes. Rare events, such as mutations, create new bands that stand out above the background pattern. Plants with bands indicative of mutations of interest were evaluated by TILLING individual members of a pool mixed with wild type DNA and then sequencing individual PCR products. Plants carrying mutations confirmed by sequencing were grown up as described above (e.g., the M2 plant could be backcrossed or outcrossed multiple times in order to eliminate background mutations and self-pollinated in order to create a plant that was homozygous for the mutation) or crossed to another plant containing WPBF mutations in a different homoeolog.

### Example 2—Genotyping and Plant Breeding of WPBF Lines

Plants carrying mutations confirmed by sequencing were grown up as described above (e.g., the M2 plant could be backcrossed or outcrossed multiple times in order to eliminate background mutations and self-pollinated in order to create a plant that was homozygous for the mutation) or crossed to another plant containing WPBF mutations in a different homoeolog. At each generation, the novel alleles were validated in the plant materials by extracting DNA, and genotyped by sequencing or by use of allele specific KASP (Kompetitive Allele Specific PCR) molecular markers (LGC Genomics, Beverly, MA) developed specifically for alleles of interest.

KASP genotyping was performed on DNA extracted from young leaves as describe in Example 1. Each reaction consisted of 5 μl master mix (KASP High-Rox Universal 2.times. Master Mix, LGC Genomics) 0.14 μl KASP Assay Mix, and 40-60 ng DNA in a total reaction volume of 10.14 The reaction mixture was then PCR amplified in a 96-well format using the following thermal cycling conditions: 94° C. for 15 minutes, then 10 cycles at 92° C. for 20 seconds followed by 61° C. for 60 seconds dropping 0.6° C. per cycle until reaching 55° C., then 35-40 cycles of 94° C. for 20 seconds followed by 55° C. for 60 seconds, and finally held at 8° C. until measurement. The subsequent reaction was evaluated at room temperature with a 7900 HT Fast Real-Time PCR system using controls of known genotypes (Applied Biosystems, Inc, Foster City, Calif, USA).

Selected plants identified with severe mutations in WPBF of the A or B or D genomes (Tables 1, 2, and 3) were crossed with other plants that contained severe mutations in WPBF in the other genomes. Severe mutations, also considered deleterious mutations, included those mutations that were predicted to have a deleterious effect on protein function by their SIFT scores (<0.05) and PSSM scores (>20), as well as those mutations that resulted in the introduction of a stop codon (nonsense mutation) or a mutation at a splice junction or a translational start codon. Severe or deleterious effects on protein function means that the protein's activity is reduced, or eliminated. In some cases the protein is undetectable. In some cases the protein is present, but inactive. Mutations that alter the amino acid sequence, but are not predicted to affect protein function are called missense mutations. Exemplary wheat plants and seeds with mutations in one or more WPBF genome are shown in Table 4. Plants and seeds with homozygous mutations are indicated by Hom, and plants and seeds with wild-type mutations are indicated with Wt.

TABLE 4
Wheat Plants and Seeds with Mutations in One or More Genomes

| Wheat | |
|---|---|
| Type | Genotype |
| Hexaploid | WPBF_A(C66Y):Hom |
| Hexaploid | WPBF_A(C66Y):Wt |
| Hexaploid | WPBF_A(C66Y):Hom, WPBF_B(W70*):Hom, |
| | WPBF_D(C63Y):Hom |
| Hexaploid | WPBF_A(C66Y):Hom, WPBF_B(W70*):Wt, |
| | WPBF_D(C63Y):Wt |
| Hexaploid | WPBF_A(C66Y):Wt, WPBF_B(W70*):Wt, |
| | WPBF_D(C63Y):Hom |
| Hexaploid | WPBF_A(M1I):Hom, WPBF_B(P59S):Hom |
| Hexaploid | WPBF_A(M1I):Hom, WPBF_B(P59S):Wt |
| Hexaploid | WPBF_B(A65V):Hom |
| Hexaploid | WPBF_B(A65V):Wt |
| Hexaploid | WPBF_B(C38Y):Hom |
| Hexaploid | WPBF_B(C63Y):Hom |
| Hexaploid | WPBF_B(G73C):Hom |
| Hexaploid | WPBF_B(G84D):Hom |
| Hexaploid | WPBF_B(P39L):Hom |
| Hexaploid | WPBF_B(P80L):Hom |
| Hexaploid | WPBF_B(P80S):Hom |
| Hexaploid | WPBF_B(Q182*):Hom |
| Hexaploid | WPBF_B(Q58*):Hom |
| Hexaploid | WPBF_B(R40W):Hom |
| Hexaploid | WPBF_B(R60C):Hom |
| Hexaploid | WPBF_B(T46I):Hom |
| Hexaploid | WPBF_B(T71I):Hom |
| Hexaploid | WPBF_B(W70*):Hom |
| Hexaploid | WPBF_B(W70*):Hom, WPBF_D(C63Y):Hom |
| Hexaploid | WPBF_B(W70*):Hom, WPBF_D(C63Y):Wt |
| Hexaploid | WPBF_B(W70*):Wt, WPBF_D(C63Y):Wt |
| Hexaploid | WPBF_B(W70R):Hom |
| Hexaploid | WPBF_D(C38Y):Hom |
| Hexaploid | WPBF_D(C41S):Hom |
| Hexaploid | WPBF_D(C49Y):Hom |
| Hexaploid | WPBF_D(C63Y):Wt |
| Hexaploid | WPBF_D(P39L):Hom |
| Hexaploid | WPBF_D(P39S):Hom |
| Hexaploid | WPBF_D(Q215*):Hom |
| Hexaploid | WPBF_D(S75F):Hom |
| Hexaploid | WPBF_D(S75F):Wt |
| Hexaploid | WPBF_D(W70*):Hom |
| Hexaploid | WPBF_D(W70*):Wt |
| Tetraploid | WPBF_A(C49Y):Wt |
| Tetraploid | WPBF_A(C49Y):Hom |
| Tetraploid | WPBF_A(G74D):Wt |
| Tetraploid | WPBF_A(G74D):Hom |
| Tetraploid | WPBF_A(G74D):Hom, WPBF_B(C38Y):Hom |
| Tetraploid | WPBF_A(G74D):Hom, WPBF_B(Q58*):Hom |
| Tetraploid | WPBF_A(G84D):Hom |
| Tetraploid | WPBF_A(G84D):Hom, WPBF_B(C38Y):Hom |
| Tetraploid | WPBF_A(G84D):Hom, WPBF_B(Q58*):Hom |
| Tetraploid | WPBF_A(G84S):Hom, WPBF_B(C38Y):Hom |
| Tetraploid | WPBF_A(G84S):Hom, WPBF_B(Q58*):Hom |
| Tetraploid | WPBF_A(M1I):Hom |
| Tetraploid | WPBF_A(M1I):Hom, WPBF_B(C38Y):Hom |
| Tetraploid | WPBF_A(M1I):Hom, WPBF_B(P59S):Hom |
| Tetraploid | WPBF_A(M1I):Hom, WPBF_B(Q58*):Hom |
| Tetraploid | WPBF_A(P80L):Hom |
| Tetraploid | WPBF_A(P80S):Hom |
| Tetraploid | WPBF_A(Q34L):Hom |
| Tetraploid | WPBF_A(W255*):Hom |
| Tetraploid | WPBF_A(W255*):Hom, WPBF_B(C38Y):Hom |
| Tetraploid | WPBF_A(W255*):Hom, WPBF_B(Q58*):Hom |
| Tetraploid | WPBF_B (T71I):Hom |
| Tetraploid | WPBF_B(C38Y):Hom |
| Tetraploid | WPBF_B(G73S):Wt |
| Tetraploid | WPBF_B(G73S):Hom |
| Tetraploid | WPBF_B(G74R):Wt |
| Tetraploid | WPBF_B(G74R):Hom |
| Tetraploid | WPBF_B(G83S):Hom |
| Tetraploid | WPBF_B(P39S):Hom |
| Tetraploid | WPBF_B(P59S):Hom |
| Tetraploid | WPBF_B(P80L):Wt |
| Tetraploid | WPBF_B(P80L):Hom |
| Tetraploid | WPBF_B(P80S):Hom |
| Tetraploid | WPBF_B(Q58*):Wt |
| Tetraploid | WPBF_B(Q58*):Hom |
| Tetraploid | WPBF_B(R77H)(E37K):Hom |
| Tetraploid | WPBF_B(S163F):Hom |
| Tetraploid | WPBF_B(T46I):Hom |
| Tetraploid | WPBF_B(T71I):Hom |

### Example 3—Analysis of Fiber Content of WPBF Mutant Lines

Fiber content was measured in wheat grains from hexaploid wheat plants grown in the field. Wheat grains having human-induced homozygous mutations in WPBF-A, WPBF-B, and WPBF-D, specifically mutations that were predicted to be deleterious (WPBF_A(C66Y):Hom, WPBF_B(W70*):Hom, and WPBF_D(C63Y):Hom) were evaluated. These mutant lines WPBF01, WPBF02, WPBF03, and WPBF09 were compared with wheat grains from sibling plants WPBF04 and WPBF05, having wild-type alleles in the three genomes and the parental un-mutated variety, Express. Both groups of plants were grown under the same conditions.

Duplicate samples of the grain from each line was analyzed for fiber and other proximate components using near infrared spectroscopy (“NIR”) and software packages supplied by the manufacturer (PerkinElmer, Inc., Waltham, MA). Averages of the measurements with standard deviations are show in Table 5. Statistical analysis between the homozygous lines and the wild-type lines was performed using the t-test.

TABLE 5
NIR Analysis of Field Grown WPBF_Grain in Express Variety

| Proximate Analysis | WPBF (3x Hom) | Wild-type (3x Wt) |
|---|---|---|
| Genotype Code | Lines WPBF01, | Lines WPBF04, and |
| | 02, 03, and 09 | 05; and Express |
| Ash (%) | 1.92 ± 0.04 | 1.96 ± 0.06 |
| Fiber, Dry basis (%) | 2.94 ± 0.78* | 2.66 ± 0.08 |
| NDF Dry basis (%) | 12.78 ± 0.60* | 11.23 ± 0.43 |
| Protein, Dry basis (%) | 16.96 ± 0.54* | 18.93 ± 0.48 |
| Starch, Dry basis (%) | 69.83 ± 0.57* | 72.84 ± 0.82 |

As shown in Table 5, an analysis of WBPF hexaploid wheat mutant lines and control lines with a NIR analysis showed no significant differences in ash content. However, both fiber and neutral detergent fiber were significantly elevated in the WPBF mutant lines compared to the wild-type controls (* indicates P<0.01). Protein and starch were reduced in the WPBF mutant lines compared to their wild-type siblings and parental line.

WPBF mutant alleles in the A, B and D genome were introgressed into a Northern Plains wheat variety background. BC1F4 lines WPBF20A and WPBF20B that were homozygous for all three mutations (WPBF_A(C66Y):Hom, WPBF_B(W70*):Hom, and WPBF_D(C63Y):Hom) produced grains that had elevated levels of fiber and neutral detergent fiber compared to grain from a wild-type sibling control line WPBF21 (Table 6). This result indicated that the human-induced WPBF mutations were responsible for the increased fiber phenotype.

TABLE 6
NIR Analysis of WPBF_Grain in
Northern Plains Variety Background

| Proximate Analysis | WPBF20A | WPBF20B | WPBF21 | WPBF21 |
|---|---|---|---|---|
| Genotype | 3xHom | 3xHom | 3xWt | 3xWt |
| Ash Dry basis (%) | 1.49 | 1.57 | 1.55 | 1.55 |
| Fiber Dry basis (%) | 3.13 | 3.14 | 2.77 | 2.78 |
| NDF Dry basis (%) | 7.63 | 8.75 | 7.47 | 7.31 |
| Protein Dry basis (%) | 15.48 | 16.32 | 16.94 | 16.90 |
| Starch Dry basis (%) | 74.39 | 72.27 | 77.13 | 77.12 |

Duplicate samples of field grown wheat grains from a nitrogen input trial were grown under 0%, 50%, 100%, and 150% additional nitrogen fertilizer application, harvested and evaluated by NIR. Average values of duplicate samples at 12% moisture basis are shown in Table 7. Fiber and Neutral detergent fiber increased with increasing nitrogen application in the WPBF mutant line, WPBF02 (3×Hom) compared to the control sibling line WPBF14 (3×Wt) and the parental variety, Express (3×Wt).

TABLE 7
NIR Analysis of WPBF Grain in Nitrogen Trial

| | Nitrogen | Yield | Ash | Protein | NDF | Fiber | Starch |
|---|---|---|---|---|---|---|---|
| Line | % | (kg) | at 12% | at 12% | at 12% | at 12% | at 12% |
| Express | 0 | 2.35 | 1.31 | 8.50 | 7.87 | 2.09 | 70.91 |
| Express | 50 | 3.70 | 1.37 | 10.04 | 7.27 | 2.07 | 70.80 |
| Express | 100 | 3.93 | 1.44 | 11.35 | 7.82 | 2.09 | 69.50 |
| Express | 150 | 4.40 | 1.50 | 12.44 | 7.88 | 2.11 | 68.88 |
| WPBF14 (Wt) | 0 | 2.08 | 1.37 | 9.96 | 7.91 | 2.12 | 70.21 |
| WPBF14 (Wt) | 50 | 3.15 | 1.43 | 10.80 | 8.17 | 2.14 | 69.37 |
| WPBF14 (Wt) | 100 | 2.78 | 1.49 | 11.93 | 8.50 | 2.17 | 68.39 |
| WPBF14 (Wt) | 150 | 4.43 | 1.52 | 13.36 | 8.72 | 2.20 | 67.52 |
| WPBF02 (Hom) | 0 | 1.95 | 1.37 | 10.27 | 8.92 | 2.28 | 67.43 |
| WPBF02 (Hom) | 50 | 3.18 | 1.38 | 11.02 | 8.87 | 2.30 | 66.66 |
| WPBF02 (Hom) | 100 | 3.60 | 1.47 | 12.30 | 9.35 | 2.40 | 65.24 |
| WPBF02 (Hom) | 150 | 4.48 | 1.47 | 13.16 | 9.50 | 2.45 | 64.46 |

Fiber components were tested on milled whole grain flour on WPBF mutant lines and their controls in two different wheat backgrounds by Medallion Labs (Minneapolis, MN, USA). Total dietary fiber including insoluble and soluble fiber components were assayed using the Fiber Rapid Integrated test (ICC DS 185). Fructans were assayed using AOAC method 997.08, and resistant starch content was measured using AOAC method 2002.02. The homozygous WPBF mutant lines had total dietary fiber, contents around 31% with increases coming from both insoluble and soluble fiber components compared to total dietary fiber contents of about 17-21% from the wild-type control lines. Absolute fructan levels were increased by around 0.5% in the Express background and 0.23% in the Northern Plains background. Surprisingly, resistant starch was elevation substantially in WPBF mutant lines from both wheat varieties.

TABLE 8
Fiber Component Analysis of Whole Grain Flour

| | Express | | N. Plains | |
|---|---|---|---|---|
| | WPBF02 | WPBF14 | WPBF20 | WPBF21 |
| Fiber Components | 3x HOM | 3x WT | 3x HOM | 3x WT |
| Total Dietary Fiber | 31.5% | 20.8% | 31.0% | 16.8% |
| Insoluble Fiber | 15.5% | 11.2% | 14.5% | 11.1% |
| Soluble dietary fiber | 8.2% | 7.4% | 10.4% | 3.5% |
| Gravimetric | | | | |
| Soluble dietary fiber | 7.8% | 2.3% | 6.1% | 2.2% |
| HPLC | | | | |
| Soluble dietary fiber | 16.0% | 9.6% | 16.5% | 5.7% |
| Total | | | | |
| Fructan | 1.66% | 1.17% | 1.01% | 0.84% |
| Resistant Starch | 13.8% | <2% | 6.5% | <2% |

Beta-glucan fiber content in WPBF mutant lines was measured using the Beta-Glucan Assay Kit (B-GLUC, Megazyme, Ireland) AOAC method 995.16 according to manufacturer's instructions. The average beta-glucan content of three WPBF mutant lines from two different experiments was similar to the beta-glucan content of two wild-type control lines (Table 9).

TABLE 9
Beta-Glucan Content of WPBF Mutant Lines and Controls

| | Beta Glucan | Average Beta Glucan |
|---|---|---|
| Sample | (%, dry basis) | (% dry basis) |
| WPBF02 (3x Hom) | 0.38%, 0.56% | 0.52% |
| WPBF03 (3x Hom) | 0.52%, 0.57% | |
| WPBF09 (3x Hom) | 0.51%, 0.59% | |
| WPBF14 (3x Wt) | 0.47%, 0.49% | 0.52% |
| WPBF05 (3x Wt) | 0.57% | |
| Barley Control (Expect 7.5%) | 7.99% | 7.99% |
| Oat Control (Expect 4.1%) | 4.30%, 3.78% | 4.0% |

Although preferred embodiments have been depicted and described in detail herein, it will be apparent to those skilled in the relevant art that various modifications, additions, substitutions, and the like can be made without departing from the spirit of the present application and these are therefore considered to be within the scope of the present application.

What is claimed:

1. A method of increasing fiber in a wheat grain, said method comprising:
providing a wheat plant or plant part comprising a Wheat Prolamin-box Binding Factor (WPBF) gene and
introducing a human-induced mutation into the WPBF gene, wherein the mutation is effective to create a wheat plant capable of producing wheat grain with increased fiber compared to wheat grain from a wheat plant not having the mutation.

2. The method of claim 1, wherein the mutation is introduced into the WPBF gene of the A genome, the B genome, or the D genome.

3. The method of claim 1, wherein the mutation is introduced into each of the WPBF genes of the A and B genomes.

4. The method of claim 1, wherein the mutation is introduced into each of the WPBF genes of the A and D genomes.

5. The method of claim 1, wherein the mutation is introduced into each of the WPBF genes of the B and D genomes.

6. The method of claim 1, wherein the mutation is introduced into each of the WPBF genes of the A, B and D genomes.

7. The method as in any of claims 1-6, in which the mutation is homozygous.

8. The method of claim 1, in which the increased fiber comprises resistant starch.

9. The method of claim 1, in which the increased fiber comprises fructan.

10. The method of claim 1, in which the increased fiber comprises soluble and insoluble fiber.

11. The method of claim 1, wherein said introducing is carried out by chemical mutagenesis.

12. The method of claim 11, wherein said chemical is EMS.

13. The method of claim 1, wherein said introducing is carried out by genome editing.

14. The method of claim 1, wherein the mutation is a single nucleotide polymorphism.

15. A method of producing dietary fiber, said method comprising:
providing a wheat plant comprising a human-induced mutation in a Wheat Prolamin-box Binding Factor (WPBF) gene, wherein said mutation causes the wheat plant to produce grain with increased fiber compared to a wheat plant without the mutation and
obtaining fiber from the grain of the wheat plant.

16. The method of claim 15, wherein the mutation is in the WPBF gene of the A genome, the B genome, or the D genome.

17. The method of claim 15, wherein the mutation is in each of the WPBF genes of the A and B genomes.

18. The method of claim 15, wherein the mutation is in each of the WPBF genes of the A and D genomes.

19. The method of claim 15, wherein the mutation is in each of the WPBF genes of the B and D genomes.

20. The method of claim 15, wherein the mutation is in each of the WPBF genes of the A, B, and D genomes.

21. The method as in any of claims 15-20, in which the mutation is homozygous.

22. The method of claim 15, wherein said obtaining comprises milling the wheat grain into whole grain flour.

23. The method of claim 15, wherein said obtaining comprises milling the wheat grain into refined flour.

24. The method of claim 15 further comprising:
incorporating the dietary fiber into a food or beverage.
